(19) United States
(12) Patent Application Publication
(10) Pub. No.: US 2024/0180871 A1
(43) Pub. Date: Jun. 6, 2024

(54) MODIFIED-RELEASE SILODOSIN COMPOSITIONS AND USE THEREOF IN METHODS FOR MALE CONTRACEPTION

(71) Applicant: PHARMAJOR INCORPORATED, Wilmington, DE (US)

(72) Inventors: Guillaume EL GLAOUI, Luxembourg (LU); Mehdi EL GLAOUI, Luxembourg (LU); Véronique AGATHON-MERIAU, Luxembourg (LU); Marguerite TULLI-CORTES, Versailles (FR); Stéphanie ANGOT, Pessac (FR); Jérôme REVEL, Pessac (FR)

(73) Assignee: PHARMAJOR INCORPORATED, Wilmington, DE (US)

(21) Appl. No.: 18/518,489

(22) Filed: Nov. 23, 2023

Related U.S. Application Data
(60) Provisional application No. 63/427,480, filed on Nov. 23, 2022.

(30) Foreign Application Priority Data
Nov. 23, 2022 (EP) 22208994.8

(51) Int. Cl. A61K 31/404; A61K 9/16; A61K 9/48

(52) U.S. Cl. CPC A61K 31/404; A61K 9/1611; A61K 9/1617; A61K 9/1635; A61K 9/1652; A61K 9/1682; A61K 9/4833

(57) ABSTRACT

The present disclosure relates to modified-release silodosin compositions including at least one pellet that comprises a silodosin drug layer and an extended-release coating.

The drug layer can comprise silodosin and a binder. The pellet can further comprise a sealing coating. The pellet can further comprise an enteric coating.

The present disclosure further relates to the use of the compositions described in methods for male contraception.

The present disclosure further relates to processes for manufacturing a pellet or dosage form as described herein.

### CROSS-REFERENCE TO RELATED APPLICATIONS

This application claims benefit under 35 U.S.C. § 119(e) of U.S. Provisional Application No. 63/427,480 filed Nov. 23, 2022, and claims benefit under 35 U.S.C. § 119(a) of European Patent Office Application No. EP 22208994.8, filed Nov. 23, 2022, the contents of which are incorporated herein by reference in their entireties.

### FIELD OF INVENTION

The technology described herein relates to compositions and methods for male contraception.

### BACKGROUND OF INVENTION

Contraceptive methods for female subjects, including contraceptive compositions, can be traced back to Middle Ages or even to Antiquity. Safer and more convenient pharmaceutical solutions for women became gradually available during the 20^{th} century and they are now part of the everyday life almost everywhere in the world. By contrast, male contraception was historically far less popular and the interest of men for drug-based contraception remained quite limited until the modern era. Although state-of-the-art male contraception methods, such as condoms or vasectomy, have well-documented drawbacks and limitations, not much effort was made to find alternative options. Thus, men willing to use oral contraception, for example to reduce the mental burden for a female partner, assume the birth control responsibility, or find an alternative to female contraception, would find very few available options as of today.

When significant investments started to be directed to this research area, the development of a male contraceptive was found to be excessively challenging. By analogy with female contraceptive compositions, most of the initial efforts were oriented towards hormone-based contraception. However, repeated administration of hormones to men is associated with psychological, behavioral, physiological, and sexual adverse effects that are generally considered inacceptable by the male subject themselves. Among those loss of sexual desire, “loss of virility” (e.g., erectile dysfunction, breast tenderness and growth of breast tissue, shrinkage of testicles and penis, or loss of muscle mass), depression, possible suicidal thoughts, decreased mental sharpness, weight gain, fatigue and hot flashes have been documented. Hormonal compositions have also significant limitations in terms of dosage amount or injection schedules. Unlike women, men do not have a hormonal cycle, which limits the development of suitable hormonal treatments for both andropause and male contraception.

Silodosin is an al-adrenoceptor antagonist with high selectivity for lower urinary tract (prostate, urethra and bladder neck) (uroselectivity), which is marketed for the treatment of signs and symptoms of benign prostatic hyperplasia (“BPH”) under trade names RAPAFLO® (United States), SILODYX® or UROREC® (European Union), among others. Alpha1-adrenergic receptors are present in arteries, smooth muscles, and central nervous system tissues. “Alpha-1-adrenoreceptor antagonists” refers to compounds inhibiting these receptors. Thereby, the hormone norepinephrine is prevented from tightening the muscles in the walls of smaller arteries and veins, so that administration of alpha-1-adrenoreceptor antagonists in humans causes the vessels to remain open and relaxed. This improves blood flow and lowers blood pressure. The male urinary smooth muscles contain high densities of alpha-1-adrenoceptors, so that alpha-1-adrenoreceptor antagonists such as silodosin can help to improve urine flow in case of prostate dysfunction, such as for example BPH.

During BPH treatment by silodosin, it was discovered that silodosin may in some cases have the effect of causing aspermia, azoospermia, or severe oligozoospermia in male subjects, in particular lack of seminal emission (aspermia) (KOBAYASHI, K. et al., International Journal of Impotence Research, June 2009, Vol. 21, pp. 306-310; SAKATA, K., et al., BMC Urology, 2012, Vol. 12, No. 29). Although they were originally considered side-effects of BPH treatment, these results suggested that silodosin can possibly be used as male contraceptive, which was further investigated (BHAT, G. et al., Indian Journal of Urology, 1 Jan. 2018, Vol. 34, No. 5, Suppl. 1, p. S7; BHAT, G. et al., World Journal of Urology, 10 May 2019, Vol. 38, pp. 747-751). However, the relevant scientific information provided by BHAT et al. remained very limited, in particular due to the experimental setting. Thus, BHAT et al. evidenced, at best, that silodosin might possibly be used in “on-demand” contraception methods. In “on-demand” contraception methods, the oral contraceptive is taken in advance, close to the sexual intercourse. This is obviously not convenient for the involved subjects. More importantly, “on-demand” contraceptives do not provide continuous contraception, even less safe and reliable continuous contraception.

Therefore, early silodosin-based contraceptive compositions did not fulfill the needs of men who need a safe contraceptive method, especially a method ensuring a continuous contraceptive effect. Moreover, a number of undesirable sex-related adverse effects are known to be associated with silodosin administration, including discomfort upon ejaculation, decrease in the quality of orgasm, decrease in erectile function, and reduction of sexual desire. Consequently, immediate release (IR) silodosin compositions known in the art, for example the compositions marketed for the treatment of BPH, were not suitable for the purpose of repositioning silodosin as a contraceptive agent.

Patent application WO 2019/180217 A1 (LABORATOIRES MAJOR) discloses the use of an extended-release (ER) formulation comprising silodosin in a non-hormonal contraception method for a male subject. In particular, WO 2019/180217 A1 discloses ER granules of (R)-silodosin identified as “formulation A”, which have the structure and composition indicated in Table 1 below.

TABLE 1
“Formulation A” of WO 2019/180217 A1

| Ingredient | Structure | % w/w |
|---|---|---|
| Cellulose microspheres | Inert core | 69.18 |
| Silodosin | Silodosin | 3.32 |
| HPMC (Opadry ©) | suspension | 3.46 |
| KH₂PO₄ | | 0.23 |
| Aquacoat ® ECD/dibutylsebaccate (DBS) | Coating | 3.81 |
| Aquacoat ® ECD/guar gum/DBS | ER coating | 20.00 |

Aquacoat® ECD is an aqueous dispersion of ethylcellulose. Guar gum is a polysaccharide, commonly used as a stabilizer or thickening agent. Dibutylsebaccate (DBS) is a dibutyl ester of sebacic acid, commonly used as plasticizer. In WO 2019/180217 A1, the ER formulation is administered once daily about the same time each day and triggers a continuous reversible aspermia, azoospermia, or severe oligozoospermia in the male subject and, interestingly, after an initial period of at least two consecutive days, the contraception is not impaired by a delay of the subsequent once daily intake. Therefore, WO 2019/180217 A1 teaches that a male contraception method using “formula A” as contraceptive composition provides safe contraception. The method of WO 2019/180217 A1 represented a significant improvement from state-of-the art methods and compositions, in particular IR silodosin compositions as used by BHAT et al.

### SUMMARY

The present disclosure provides further improvements to the physico-chemical and/or biological properties of modified-release silodosin formulations for a male oral contraceptive, including improvements that address the chemical stability of silodosin, intra-batch homogeneity, inter-batch reproducibility, migration of silodosin within the modified-release granule, stability over time of the release profile (e.g., dissolution profile), adverse effects (in particular sex-related effects), and reliability of the contraception in relation to the subject's compliance. Also provided herein are compositions permitting easy determination or adjustment of the optimal dose of silodosin (“dose-ranging”) and/or release rate of silodosin. In particular, compositions described herein prevent or limit the “burst release effect,” which is the unwanted and uncontrolled, early and fast, release of the active ingredient. Improvements in regard to manufacturing process aspects also optimize parameters, e.g., in terms of degradation of silodosin and coating yield, among others.

Described herein are additional modified-release compositions, the modified-release effect being obtained by means of at least one pellet that comprises an inert core, at least one drug layer applied to the inert core, the drug layer comprising silodosin and at least one binder, and at least one extended-release coating surrounding the drug layer or the optional sealing coating. In certain embodiments, the pellet can further comprise at least one sealing coating surrounding the drug layer. In other embodiments, the pellet can further comprise at least one enteric coating comprising at least one polymer selected from acrylate polymers, cellulose polymers, and mixtures thereof. In the technology described herein, the extended-release coating comprises at least one vinyl polymer, which was surprisingly identified by the Applicant as a very relevant material for preparing modified-release silodosin compositions. In particular, the use of vinyl polymers unexpectedly overcome some significant limitations of cellulose-based ER coatings (e.g., ethylcellulose) for silodosin formulation as a contraceptive.

In one aspect, described herein is a pellet comprising: (a) an inert core; (b) at least one drug layer applied to the inert core, the drug layer comprising: silodosin and at least one binder; and (c) at least one extended-release coating surrounding the drug layer, wherein the extended-release coating comprises at least one vinyl polymer.

In one embodiment of this or any other aspect described herein, the pellet further comprises at least one sealing coating surrounding the drug layer, and wherein the extended-release coating surrounds the sealing coating.

In another embodiment of this or any other aspect described herein, the binder comprises a cellulose polymer. In another embodiment, the cellulose polymer comprised by the binder is selected from the group consisting of hydroxypropyl cellulose (HPC), hydroxypropylmethylcellulose (HPMC), carboxymethylcellulose, methylcellulose, ethyl cellulose, povidone, polyvinylpyrrolidone, and mixtures thereof.

In another embodiment of this or any other aspect described herein, the sealing coating comprises at least one cellulose polymer. In another embodiment of this or any other aspect described herein the cellulose polymer comprised by the sealing coating is selected from the group consisting of hydroxypropyl methylcellulose (HPMC), carboxymethylcellulose, methylcellulose, ethyl cellulose, and mixtures thereof.

In another embodiment of this or any other aspect described herein, the inert core comprises a cellulose polymer or a mixture thereof. In another embodiment of this or any other aspect described herein, the inert core comprises microcrystalline cellulose.

In another embodiment of this or any other aspect described herein, the inert core has a particle size ranging from about 300 to 500 μm.

In another embodiment of this or any other aspect described herein, the extended-release coating comprises at least one polyvinyl ester polymer. In another embodiment of this or any other aspect described herein, the polyvinyl ester polymer is a polyvinyl acetate polymer. In another embodiment of this or any other aspect described herein, the polyvinyl acetate polymer is polyvinyl acetate (PVA). In another embodiment of this or any other aspect described herein, the extended-release coating further comprises povidone (PVP). In another embodiment of this or any other aspect described herein, the extended-release coating comprises about 90% w/w of polyvinyl acetate (PVA) and about 9% w/w of povidone (PVP), in weight by weight of the extended-release coating.

In another embodiment of this or any other aspect described herein, the pellet further comprises one or more of: one or more antioxidant; one or more anti-tacking and/or charge agent; and/or one or more plasticizer. In another embodiment of this or any other aspect described herein, the antioxidant is selected from phenols, vitamin E and derivatives thereof, vitamin C and derivatives thereof, propyl gallate, and mixtures thereof. In another embodiment of this or any other aspect described herein, the antioxidant is selected from butylated hydroxytoluene (BHT), butylated hydroxyanisole (BHA), alpha-tocopherol, ascorbyl palmitate, propyl gallate, and mixtures thereof. In another embodiment of this or any other aspect described herein, the anti-tacking and/or charge agent is selected from inorganic carbonates, magnesium silicates, and mixtures thereof. In another embodiment of this or any other aspect described herein, the anti-tacking and/or charge agent is selected from calcium carbonate (CaCO₃), talc, and mixtures thereof. In another embodiment of this or any other aspect described herein, the plasticizer is a citric acid ester. In another embodiment of this or any other aspect described herein, the plasticizer is triethyl citrate (TEC).

In another embodiment of this or any other aspect described herein, the pellet further comprises: at least one enteric coating, wherein the enteric coating is either surrounding the extended-release coating, or surrounded by the extended-release coating and surrounding the sealing coating or the drug layer.

In another embodiment of this or any other aspect described herein, the enteric coating comprises at least one acrylate copolymer. In another embodiment of this or any other aspect described herein, the acrylate copolymer is a methacrylic acid ethylacrylate (MAE) copolymer.

In another embodiment of this or any other aspect described herein, the pellet comprises: a pellet A comprising or consisting essentially of: from about 24 to 95% w/w of the inert core; from about 5 to 76% w/w of the drug layer, the drug layer comprising: from about 5 to 25% w/w of the silodosin; from about 0.1 to 7.5% w/w of the binder; from about 0 to 20% w/w of one or more antioxidant; and from about 0 to 25% w/w of one or more anti-tacking and/or charge agent, in weight relative to the total weight of the pellet A.

In another embodiment of this or any other aspect described herein, the pellet A is comprised in a pellet B comprising or consisting essentially of: from about 90 to 100% w/w of pellet A; and from about 0 to 10% w/w of one or more sealing coating, in weight relative to the total weight of the pellet B.

In another embodiment of this or any other aspect described herein, the pellet A or pellet B is comprised in a pellet C comprising or consisting essentially of: from about 50 to 98% w/w of pellet A or pellet B, and an extended-release coating comprising: from about 0.5 to 47% w/w of at least one extended-release agent; from about 0.02 to 7% w/w of at least one plasticizer; and from about 0.3 to 23% w/w of at least one anti-tacking agent, in weight relative to the total weight of the pellet C.

In another embodiment of this or any other aspect described herein, the pellet C is comprised in a pellet D consisting essentially of: from about 50 to 95% w/w of the pellet C, and an enteric coating comprising: from about 4 to 49.5% w/w of at least one enteric agent; and from about 0.04 to 10% w/w of at least one plasticizer, in weight relative to the total weight of the pellet D.

In another embodiment of a pellet as described herein: (a) the drug layer is applied onto the inert core at a weight gain ranging from about 5 to 318%; (b) the sealing coating agent is applied onto the drug layer at a weight gain ranging from 0 to about 11%; (c) the extended-release coating agent is applied onto the drug layer or the sealing coating at a weight gain ranging from about 2 to 100%; and/or (d) the enteric coating agent is applied onto the extended-release coating at a weight gain ranging from 0 to about 100%.

In another embodiment of this or any other aspect described herein, the pellet comprises: (a) an inert core comprising cellulose microspheres; (b) at least one drug layer applied to the inert core, wherein the drug layer comprises: silodosin, hydroxypropyl cellulose (HPC), calcium carbonate (CaCO₃), and butylated hydroxytoluene (BHT); (c) at least one sealing coating surrounding the drug layer, wherein the sealing coating comprises hydroxypropyl methylcellulose (HPMC); and (d) at least one extended-release coating surrounding the drug layer or the optional sealing coating, wherein the extended-release coating comprises: polyvinyl acetate (PVA), polyvinylpyrrolidone (PVP), triethyl citrate (TEC), and talc.

In another embodiment of this or any other aspect described herein, the pellet further comprises: (e) at least one enteric coating, wherein the enteric coating is either surrounding the extended-release coating, or surrounded by the extended-release coating and surrounding the optional sealing coating or the drug layer, wherein the enteric coating comprises: a methacrylic acid ethylacrylate copolymer 1:1 (MAE 1:1), and triethyl citrate (TEC).

In another aspect, described herein is a pharmaceutical composition comprising a plurality of pellets as described herein. In one embodiment of this aspect or any other aspect described herein, the plurality of pellets is comprised in a capsule. In one embodiment of this aspect or any other aspect described herein, the capsule is a hard-shell capsule. In one embodiment of this aspect or any other aspect described herein, the capsule is a functional capsule. In another embodiment of this aspect or any other aspect described herein, the functional capsule comprises an enteric capsule.

In one embodiment of this aspect or any other aspect described herein, the plurality of pellets comprises an amount of the silodosin ranging from about 4 to 32 mg. In another embodiment of this aspect or any other aspect described herein, the plurality of pellets comprises an amount of the silodosin ranging from about 8 to 28 mg. In another embodiment of this aspect or any other aspect described herein, the plurality of pellets comprises an amount of the silodosin ranging from about 12 to 24 mg.

In another aspect, described herein is a contraception method for a male subject comprising a step of administration of a pharmaceutical composition as described herein to the male subject at about the same time each day. In one embodiment, the administration is oral administration. In one embodiment, the contraception method provides continuous, reversible aspermia, azoospermia, or severe oligozoospermia in a male subject sufficient for contraceptive effect.

In another aspect, described herein is a process for manufacturing a plurality of pellets as described herein, or a pharmaceutical composition as described herein, wherein the process comprises the following steps: (1-a) preparing a drug solution or drug suspension comprising a drug solution comprising silodosin, at least one binder and at least one solvent; (1-b) applying the drug solution or drug suspension onto a plurality of inert cores, thereby obtaining a plurality of pellets A; (2-a) applying a sealing coating suspension onto the plurality of pellets A, thereby obtaining a plurality of pellets B; (3-a) preparing an extended-release coating suspension comprising at least one extended-release coating agent, at least one plasticizer, and at least one anti-tacking agent; then (3-b) applying the extended-release coating suspension onto the plurality of pellets A or onto the plurality of pellets B, thereby obtaining the plurality of pellets as described herein. In one embodiment of this aspect or any other aspect described herein, a step of preparing a sealing coating suspension comprising at least one sealing coating agent is performed before step (2-a). In another embodiment of this aspect or any other aspect described herein, the drug solution of step (1-a) contains at least one antioxidant. In another embodiment of this aspect or any other aspect described herein, the drug solution of step (1-a) contains at least at least one anti-tacking and/or charge agent.

In another aspect, described herein is a process for manufacturing a plurality of pellets as described herein, or a pharmaceutical composition as described herein, wherein the process comprises the following steps: (1-a) preparing a drug solution or drug suspension comprising a drug solution comprising silodosin, at least one binder and at least one solvent; (1-b) applying the drug solution or drug suspension onto a plurality of inert cores, thereby obtaining a plurality of pellets A; (2-a) applying a sealing coating suspension onto the plurality of pellets A, thereby obtaining a plurality of pellets B; (3-a) preparing an extended-release coating suspension comprising at least one extended-release coating agent, at least one plasticizer, and at least one anti-tacking agent; then (3′-b) applying the extended-release coating suspension onto the plurality of pellets A or onto the plurality of pellets B, thereby obtaining a plurality of pellets C; (4′-a) preparing an enteric coating suspension comprising at least one enteric coating agent and at least one plasticizer; then (4′-b) applying the enteric coating suspension onto the plurality of pellets C, thereby obtaining the plurality of pellets as described herein. In one embodiment of this aspect or any other aspect described herein, a step of preparing a sealing coating suspension comprising at least one sealing coating agent is performed before step (2-a). In another embodiment of this aspect or any other aspect described herein, the drug solution of step (1-a) contains at least one antioxidant. In another embodiment of this aspect or any other aspect described herein, the drug solution of step (1-a) contains at least at least one anti-tacking and/or charge agent.

In another aspect, described herein is a process for manufacturing a plurality of pellets as described herein or a pharmaceutical composition as described herein, wherein the process comprises the following steps: (1-a) preparing a drug solution or drug suspension comprising a drug solution comprising silodosin, at least one binder and at least one solvent; (1-b) applying the drug solution or drug suspension onto a plurality of inert cores, thereby obtaining a plurality of pellets A; (2-a) applying a sealing coating suspension onto the plurality of pellets A, thereby obtaining a plurality of pellets B; (3″-a) preparing an enteric coating suspension comprising at least one enteric coating agent and at least one plasticizer; then (3″-b) applying the enteric coating suspension onto the plurality of pellets A or onto the plurality of pellets B, thereby obtaining a plurality of pellets E; (4″-a) preparing an extended-release coating suspension comprising at least one extended-release coating agent, at least one plasticizer, and at least one anti-tacking agent; then (4″-b) applying the extended-release coating suspension onto the plurality of pellets E, thereby obtaining the plurality of pellets as described herein. In one embodiment of this or any other aspect described herein, a step of preparing a sealing coating suspension comprising at least one sealing coating agent is performed before step (2-a). In another embodiment of this aspect or any other aspect described herein, the process further comprises a step of filling of the obtained plurality of pellets into at least one capsule, thereby obtaining a pharmaceutical composition as described herein. In another embodiment of this aspect or any other aspect described herein, the drug solution or drug suspension of step (1-a) contains at least one antioxidant. In another embodiment of this aspect or any other aspect described herein, the drug solution or drug suspension of step (1-a) contains at least at least one anti-tacking and/or charge agent.

The technology described herein relates to a pellet comprising: (a) an inert core; (b) at least one drug layer applied to the inert core, the drug layer comprising: silodosin and at least one binder; (c) optionally, at least one sealing coating surrounding the drug layer; and/or (d) at least one extended-release coating surrounding the drug layer or the optional sealing coating, wherein the extended-release coating comprises at least one vinyl polymer.

According to some embodiments, the binder is selected from cellulose polymers; preferably the binder is selected from hydroxypropyl cellulose (HPC), hydroxypropylmethylcellulose (HPMC), carboxymethylcellulose, methylcellulose, ethyl cellulose, povidone, polyvinylpyrrolidone, and mixtures thereof.

According to some embodiments, the optional sealing coating comprises at least one cellulose polymer; preferably the cellulose polymer is selected from hydroxypropyl methylcellulose (HPMC), carboxymethylcellulose, methylcellulose, ethyl cellulose, and mixtures thereof.

According to some embodiments, the inert core comprises cellulose polymers and mixtures thereof; preferably the inert core comprises microcrystalline cellulose; and/or the inert core has a particle size ranging from about 300 to 500 μm.

According to some embodiments, the extended-release coating comprises at least one polyvinyl ester polymer, preferably a polyvinyl acetate polymer, more preferably polyvinyl acetate (PVA).

According to some embodiments, the pellet further comprises at least one antioxidant; preferably the antioxidant is selected from phenols, vitamin E and derivatives thereof, vitamin C and derivatives thereof, propyl gallate, and mixtures thereof; more preferably the antioxidant is selected from butylated hydroxytoluene (BHT), butylated hydroxyanisole (BHA), alpha-tocopherol, ascorbyl palmitate, propyl gallate, and mixtures thereof; at least one anti-tacking and/or charge agent; preferably the anti-tacking and/or charge agent is selected from inorganic carbonates, magnesium silicates, and mixtures thereof; more preferably the anti-tacking and/or charge agent is selected from calcium carbonate (CaCO₃), talc, and mixtures thereof; and/or at least one plasticizer; preferably the plasticizer is selected from citric acid esters; more preferably the plasticizer is triethyl citrate (TEC).

According to some embodiments, the pellet further comprises: (e) at least one enteric coating, wherein the enteric coating is either surrounding the extended-release coating, or surrounded by the extended-release coating and surrounding the optional sealing coating or the drug layer. In some embodiments, the enteric coating comprises at least one acrylate copolymer, preferably a methacrylic acid ethylacrylate (MAE) copolymer.

According to some embodiments, the pellet comprises:
a pellet A comprising or consisting essentially of from about 24 to 95% w/w of the inert core, from about 5 to 76% w/w of the drug layer, the drug layer comprising: from about 5 to 25% w/w of the silodosin, from about 0.1 to 7.5% w/w of the binder, from about 0 to 20% w/w of one or more antioxidant, and from about 0 to 25% w/w of one or more anti-tacking and/or charge agent, in weight relative to the total weight of the pellet A;
wherein the pellet A is optionally comprised in a pellet B comprising or consisting essentially of from about 90 to 100% of the pellet A, and from about 0 to 10% w/w of one or more sealing coating surrounding the drug layer, in weight relative to the total weight of the pellet B;
wherein the pellet A or the optional pellet B is comprised in a pellet C comprising or consisting essentially of from about 50 to 98% w/w of the pellet A or the optional pellet B, and an extended-release coating comprising: from about 0.5 to 47% w/w of at least one extended-release agent, from about 0.02 to 0.7% w/w of at least one plasticizer, and from about 0.3 to 23% w/w of at least one anti-tacking agent; in weight relative to the total weight of the pellet C; and
wherein the pellet C is optionally comprised in a pellet D comprising or consisting essentially of from about 50 to 95% w/w of the pellet C, and an enteric coating comprising: from about 4 to 49.5% w/w of at least one enteric agent, and from about 0.04 to 10% w/w of at least one plasticizer, in weight relative to the total weight of the optional pellet D.

According to some embodiments, (b) the drug layer is applied onto the inert core at a weight gain ranging from about 5 to 318%, (c) the optional sealing coating agent is applied onto the drug layer at a weight gain ranging from 0 to about 11%, (d) the extended-release coating agent is applied onto the drug layer or the optional sealing coating at a weight gain ranging from about 2 to 100%, and/or (e) the optional enteric coating agent is applied onto the extended-release coating at a weight gain ranging from 0 to about 100%.

The technology described herein further relates to a dosage form comprising a plurality of pellets as described herein. According to some embodiments, the plurality of pellets is comprised in a capsule, preferably a hard-shell capsule and/or a functional capsule (e.g., an enteric capsule). According to some embodiments, the plurality of pellets comprises an amount of the silodosin ranging from about 4 to 32 mg, preferably ranging from about 8 to 28 mg, more preferably ranging from about 12 to 24 mg.

The technology described herein further relates to a contraception method for a male subject comprising a step of administration of a dosage form as described herein to the male subject at about the same time each day.

The technology described herein further relates to a process for manufacturing a plurality of pellets as described herein or a dosage form as described herein, wherein the process comprises the following steps: (1-a) preparing a drug solution or drug suspension comprising a drug solution comprising silodosin, at least one binder, at least one solvent, and, optionally, at least one antioxidant; and optionally, at least one anti-tacking and/or charge agent, then (1-b) applying the drug solution or drug suspension onto a plurality of inert cores, thereby obtaining a plurality of pellets A; (2-a) optionally, preparing a sealing coating suspension comprising at least one sealing coating agent; then (2-b) applying the sealing coating suspension onto the plurality of pellets A, thereby obtaining a plurality of pellets B; (3-a) preparing an extended-release coating suspension comprising at least one extended-release coating agent, at least one plasticizer, and at least one anti-tacking agent; then (3-b) applying the extended-release coating suspension onto the plurality of pellets A or onto the plurality of optional pellets B, thereby obtaining the plurality of pellets as described herein; or (3′-a) preparing an extended-release coating suspension comprising at least one extended-release coating agent, at least one plasticizer, and at least one anti-tacking agent; then (3′-b) applying the extended-release coating suspension onto the plurality of pellets A or onto the plurality of optional pellets B, thereby obtaining a plurality of pellets C; (4′-a) preparing an enteric coating suspension comprising at least one enteric coating agent and at least one plasticizer; then (4′-b) applying the enteric coating suspension onto the plurality of pellets C, thereby obtaining the plurality of pellets as described herein; or (3″-a) preparing an enteric coating suspension comprising at least one enteric coating agent and at least one plasticizer; then (3″-b) applying the enteric coating suspension onto the plurality of pellets A or onto the plurality of optional pellets B, thereby obtaining a plurality of pellets E; (4″-a) preparing an extended-release coating suspension comprising at least one extended-release coating agent, at least one plasticizer, and at least one anti-tacking agent; then (4″-b) applying the extended-release coating suspension onto the plurality of pellets E, thereby obtaining the plurality of pellets as described herein; and (5) optionally, filling of the obtained plurality of pellets into at least one capsule, thereby obtaining the dosage form as described herein.

### Definitions

In the present disclosure, the following terms have the following meanings, unless otherwise indicated.

“About” is used herein to mean approximately, roughly, around, or in the region of. When the term “about” is preceding a figure, it means plus or less (“plus or minus” or “more or less”) 10% of the value of said figure. When the term “about” is used in conjunction with a numerical range, it modifies that range by extending the boundaries above and below the numerical values set forth by 10%.

“Active ingredient” or “active pharmaceutical ingredient” or “drug” (in short “API”) are synonyms and refer collectively to contraceptive agents, therapeutic agents, and agents that are both contraceptive and therapeutic agents.

“Administration”, or a variant thereof (e.g., “administering”), means providing an active ingredient alone or as part of a pharmaceutically acceptable composition, to the subject in whom/which the contraception is to be provided.

“Aspermia” refers to inability to produce or ejaculate semen.

“Azoospermia” refers to the absence of sperm in the semen.

“Binder” or “binding agent” refers to a substance that holds or draws other substances or materials together to form a cohesive whole mechanically, chemically, by adhesion or cohesion.

“Coating agent” refers to an agent that, when applied to a surface of a substrate and, when needed, submitted to a finalization step (e.g., curing, setting, polymerization or crosslinking), will result in a “coating material” (in short “coating”) covering the surface of the substrate.

“Contraception” refers to prophylactic or preventative measures, wherein the object is to prevent or at least reduce the risk of pregnancy. In the technology described herein, the objective of contraception is to prevent fertilization, i.e., avoiding the fusion of the gametes (e.g., the union of a human egg and sperm). Those in need of contraception are typically subjects of childbearing age, regardless of their fertility status, who wish to avoid pregnancy. While contraception may be irreversible or reversible, many subjects prefer that it is reversible. When contraception is achieved by making the male subject unable to conceive, it is referred to as “male contraception” (or “male subject contraception”). In preferred embodiments, the contraception comprises triggering a continuous aspermia, azoospermia, or severe oligozoospermia in a male subject. In these embodiments, contraception is male contraception.

“Contraceptive agent” refers to a compound for contraceptive use and relating to birth control. Especially, a contraceptive agent may be indicated for preventing pregnancy. A contraceptive agent can also be indicated for improving the contraceptive activity of another contraceptive agent.

“Contraceptive composition” (in short “contraceptive”) refers to a composition for contraceptive use and relating to birth control. Especially, a contraceptive composition may be indicated for preventing pregnancy. A contraceptive composition can also be indicated for improving the contraceptive activity of another contraceptive composition.

“Contraceptive efficacy” or “contraceptively effective” refers to the function of a treatment as described herein for inducing reversible aspermia, azoospermia or severe oligozoospermia in which a male subject's ejaculate is incapable of inducing pregnancy through sexual contact in a female sexual partner. Accordingly, the term “sufficient for contraceptive effect,” when used, e.g., in reference to a treatment regimen or an amount of a drug or formulation, refers to a treatment that renders a male subject's ejaculate incapable of inducing pregnancy through sexual contact in a female sexual partner. “Interrupting” contraceptive efficacy refers to the return of a male subject's capacity to impregnate a female sexual partner through sexual contact.

“Does not affect the continuous state of aspermia, azoospermia or severe oligozoospermia sufficient for contraceptive effect,” when used in reference to failure to administer one or more daily doses of a formulation as described herein (such as, for example, a delay of the subsequent once daily intake), e.g. after an initial period of regular dosing sufficient for contraceptive effect, means that the failure to administer a given dose or doses over a given span of time (such as, for example, a delay of the subsequent once daily intake) after contraceptive efficacy is established does not interrupt the contraceptive efficacy of the treatment over that span of time.

“Contraception method” refers to a method whose objective is to induce or maintain contraception in a subject. A contraception method can include the definition of at least one subject and/or administration scheme.

“Delayed release” (in short “DR”) refers the release of an active ingredient (e.g., silodosin) from a composition wherein the active ingredient (API) is not released immediately after administration to a subject, by contrast with an immediate release (IR) composition comprising the same API in the same dosage. A “delayed-release composition” is a composition that is suitable for obtaining a DR release of the API comprised therein, after its administration to a subject. A “delayed-release coating” is a coating that contributes to render a composition “delayed-release” as defined herein. The European Pharmacopeia defines “delayed release” as: “A delayed-release dosage form is a modified-release dosage form, usually administered orally, where the onset of release of the active substance(s) is adjusted to take place after a specific time or at a specific location in the gastrointestinal tract. Delayed release is achieved by a special formulation design and/or manufacturing method. Delayed-release dosage forms include gastro-resistant preparations, which is consistent with the above definitions.

“Enteric coating” refers to a barrier, typically a polymer coating, which is applied onto an oral medication and prevents its dissolution or disintegration in the gastric environment. Enteric coatings are useful either in protecting drugs from the acidity of the stomach, protecting the stomach from the detrimental effects of the drug, or to release the drug after the stomach (usually in the upper tract of the intestine). Typically, an enteric coating is expected to dissolve at a pH equal or above about 5.5. In particular, an enteric coating can contribute to avoid or decrease the impact of the acidic environment of the stomach on the release rate of the active ingredient. Based on this last category of effects, enteric coatings can be considered a specific type of “delayed-release” coating. An “enteric agent” refers to an agent that, when applied to a surface of a substrate and, when needed, submitted to a finalization step (e.g., curing, setting, polymerization or crosslinking), will result in a “enteric coating” (as defined herein) covering the surface of the substrate.

“Extended-release” (in short “ER”) refers the release of an active ingredient (e.g., silodosin) from a composition wherein the active ingredient (API) is released over time after administration to a subject, by contrast with an immediate release (IR) composition comprising the same API in the same dosage. An “extended-release composition” is a pharmaceutical composition that is suitable for obtaining an ER release of the API comprised therein, after its administration to a subject. An “extended-release coating” is a coating that contributes to render a composition “extended-release” as defined herein. An “extended-release agent” refers to an agent that, when applied to a surface of a substrate and, when needed, submitted to a finalization step (e.g., curing, setting, polymerization or crosslinking), will result in a “extended-release coating” (as defined herein) covering the surface of the substrate.

“Human” refers to a male or female human subject at any stage of development, including neonate, infant, juvenile, adolescent and adult. In some preferred embodiments, the human is a male subject. In some preferred embodiments, the human is an adolescent or adult subject.

“Modified-release” (in short “MR”) refers to the release of an active ingredient (e.g., silodosin) from a composition that includes at least one modification of the release of the active ingredient (API) after administration to a subject, when compared to an immediate release (IR) composition comprising the same API in the same dosage. According to some embodiments, “modified-release” means “extended-release (ER)” associated with at least one modification of the release of the API. In some embodiments, “modified-release” means “extended-release (ER)” and “delayed release (DR)”, namely, a release that is both extended and delayed (DR+ER). A “modified-release composition” is a pharmaceutical composition that is suitable for obtaining a modified release of the API comprised therein, after its administration to a subject. A “modified-release coating” is a coating that contributes to render a composition “modified-release” as defined herein.

“Pellet” or “granule” refers to a small, compressed, hard chunk of matter. Typically, a pellet comprises a core surrounded by a plurality of successive coatings.

“Pharmaceutical composition” refers to a composition comprising at least one active ingredient (e.g., silodosin) and at least one pharmaceutically acceptable carrier. Thus, contraceptive compositions can be “pharmaceutical compositions” in the sense of the present disclosure.

“Pharmaceutically acceptable” means that the ingredients of a composition are compatible with each other and not deleterious to the subject to whom/which it is to be administered.

“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA. Examples of pharmaceutically acceptable carriers include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

“Plasticizer” or “plasticizing agent” refers to a substance that is added to a material to make it softer and more flexible, to increase its plasticity, to decrease its viscosity, and/or to decrease friction during its handling in manufacture.

“Polymer” preceded by a term referring to a general class of polymers (e.g., “acrylate polymer”) refers to any homopolymers, copolymers, and mixtures thereof belonging to indicated class. For example, a reference to “acrylate polymers” encompasses “acrylate homopolymers”, “acrylate copolymers”, and any mixture thereof. Copolymers can be block, alternated and/or randomized. Copolymers can also include monomers that do not belong to the indicated class but are polymerizable by the same routes.

“Prodrug” refers to a pharmacologically acceptable derivative of an active ingredient (e.g., silodosin) whose in vivo biotransformation product is the active ingredient (active drug). Prodrugs are typically characterized by increased bioavailability and are readily metabolized in vivo into the active compounds. Non-limiting examples of prodrugs include amide prodrugs and carboxylic acid ester prodrugs.

“Release profile” for a drug containing composition refers to the amount of drug released from the composition over time under a given set of conditions. A release profile can be expressed as a curve showing drug released (e.g., into solution, or as measured in plasma, for example) versus time. A “stable” drug release profile is one in which the drug release profile remains substantially the same over time in storage for the drug containing composition. By “substantially the same” in this context is meant that the value for drug released at a given time in solution for the composition after storage for a given time is within 10% (e.g., +/−10%, +/−9%, +/−8%, +/−7%, +/−6%, +/−5%, +/−4%, +/−3%, +/−2%, or +/−1%) of the value at that given time in solution prior to the storage. An “unstable” release profile is one in which the drug release profile does not remain substantially the same over time in storage (i.e., differs by more than 10%, e.g., more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90% or more at a given time) for the drug containing composition.

“Severe oligozoospermia” refers to semen with a low total number or low concentration of sperm, typically about less than or equal to 5×10⁶ sperms per ejaculate, preferably about less than or equal to 1×10⁶ sperms per ejaculate.

“Solvate” refers to a molecular complex comprising a compound (e.g., silodosin) and contains stoichiometric or sub-stoichiometric amounts of one or more pharmaceutically acceptable solvent molecule such as ethanol. The term “hydrate” refers to when said solvent is water.

“Subject” refers to an animal, typically a warm-blooded animal, in particular a mammal, more specifically a primate, preferably a human. The subject can be a “patient”. Subjects can be referred to as “male subject(s)” or “female subject(s)” depending on their respective sex. In preferred embodiments, the subject is a male subject. In preferred embodiments, the subject is in need of a contraception.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

### BRIEF DESCRIPTION OF THE DRAWINGS

FIG. 1 is a drawing showing the main steps of the manufacture of pellets [Pellets A, B, C and D] and dosage forms [Formulations (I), (III), (V) and (VI)] as described herein. References: inert core (a), drug layer comprising silodosin (b), optional sealing coating (c), extended-release coating (d), optional enteric coating (e), conventional capsule (f), functional enteric capsule (g).

FIG. 2 is a graph showing the dissolution profile at pH 6.8 of the modified-release Formulation (I-a) (black curve with diamond marks, on the right) compared to the dissolution profile of an immediate release (IR) formulation (grey curve with circular marks, on the left).

FIG. 3 is a graph showing the dissolution profile of Formulation I-a in HCl 0.1 N solution (black curve with circular marks, at the top) and in a pH 6.8 medium (grey curve with diamond marks, at the bottom).

FIG. 4 is a graph showing the dissolution profile of Formulation (I-a) in a pH 6.8 medium at T₀ (black curve with diamond marks, in the center), after 3 months of storage at 40° ° C. and 75% relative humidity (RH) (grey curve with triangle marks, at the bottom), after 9 months of storage at 25° C. and 60% RH (grey curve with square marks, at the top), and after 18 months of storage at 25° C. and 60% RH (dark curve with circular marks, at the top).

FIG. 5 is a graph showing the dissolution profile at pH 6.8 of the modified-release Formulation (V-a) (black curve with diamond marks, on the right) compared to the dissolution profile of an immediate release (IR) formulation (grey curve with circular marks, on the left).

FIG. 6 is a graph showing the dissolution profile of Formulation (V-a) in a progressive pH medium at T₀ (black curve with diamond marks, at the top), after 3 months of storage at 40° C. and 75% relative humidity (RH) (grey curve with triangle marks, at the bottom), and after 9 months of storage at 25° C. and 60% RH (grey curve with square marks, at the top).

FIG. 7 is a graph showing the dissolution profile of Comparative Formulation (VII-a) in a 0.1 N HCl solution (black curve with circular marks) and in a pH 6.8 medium (grey curve with square marks).

FIG. 8 is a graph showing the dissolution profile of Comparative Pellets (VII-b) in a 0.1 N HCl solution at T₀ (black curve with diamond marks, at the top), after 2 months of storage at 25° ° C. and 60% RH (grey curve with square marks, in the center), and after 2 months of storage at 40° C. and 75% relative humidity (RH) (grey curve with triangle marks, at the bottom).

### DETAILED DESCRIPTION

Described herein are compositions and methods relating to improved male oral contraceptive formulations comprising silodosin, methods of making them, and methods of using them for male contraception. The following provides considerations for one of ordinary skill in the art to make and use the subject compositions.

### Pellet

One object of the technology described herein is a pellet comprising:
(a) an inert core;
(b) at least one drug layer applied to the inert core, the drug layer comprising silodosin and at least one binder;
(d) at least one extended-release coating surrounding the drug layer, wherein the extended-release coating comprises at least one vinyl polymer.

According to some embodiments, the pellet has a nearly-spherical or spherical shape, i.e., the pellet is “spherical” or is a “sphere”. In some embodiments, the sphere is filled sphere (or “ball”).

### Inert Core

“Inert core” means that the core is chemically inert (or “neutral”). Thus, it does not react with active ingredients. The core can be inert, for example, because of the nature of its material or by the presence of an isolating coating.

According to some embodiments, the inert core has a nearly-spherical or spherical shape, i.e., the inert core is “spherical” or is a “sphere”. In some embodiments, the sphere is an “empty” sphere. In some embodiments, the sphere is a “filled” sphere (or “ball”).

According to some embodiments, the inert core has a particle size ranging from about 106 to 850 μm (85% or more of the particles fall in this range). In some embodiments, the inert core has a particle size ranging from about 106 to 212 μm (at least 85% of the particles fall in this range). In some embodiments, the inert core has a particle size ranging from about 150 to 300 μm (at least 85% of the particles fall in this range). In some embodiments, the inert core has a particle size ranging from about 300 to 500 μm (at least 85% of the particles fall in this range). In some embodiments, the inert core has a particle size ranging from about 500 to 710 μm (at least 85% of the particles fall in this range). In some embodiments, the inert core has a particle size ranging from about 710 to 850 μm (at least 85% of the particles fall in this range). In some preferred embodiments, the inert core has a particle size ranging from about 300 to 500 μm (at least 85% of the particles fall in this range).

According to some embodiments, the inert core has a particle size ranging from about 100 to 1400 μm (at least 85% of the particles fall in this range). In some embodiments, the inert core has a particle size ranging from about 100 to 200 μm (at least 85% of the particles fall in this range). In some embodiments, the inert core has a particle size ranging from about 200 to 355 μm (at least 85% of the particles fall in this range). In some embodiments, the inert core has a particle size ranging from about 350 to 500 μm (at least 85% of the particles fall in this range). In some embodiments, the inert core has a particle size ranging from about 500 to 710 μm (at least 85% of the particles fall in this range). In some embodiments, the inert core has a particle size ranging from about 700 to 1000 μm (at least 85% of the particles fall in this range). In some embodiments, the inert core has a particle size ranging from about 1000 to 1400 μm (at least 85% of the particles fall in this range).

“Particle size” refers to the particle width measure by sieve analysis. A sieve stack consists of several sieves with increasing aperture. The sample is placed on the uppermost sieve and the stack is shaken. As a result, the particles are distributed to the sieves in the stack according to their size.

The Applicant unexpectedly observed that, when manufacturing silodosin compositions, the yield of the step of application of the drug layer (silodosin) on the inert core was high enough for industrial needs. The Applicant surprisingly found out that the use of an inert core having an average particle size ranging from about 300 to 500 μm can significantly increase the coating performance of the drug layer on the inert core (up to 95%).

According to some embodiments, the inert core comprises cellulose polymers and mixtures thereof. In some specific embodiments, the inert core comprises microcrystalline cellulose (MCC). In some preferred embodiments, the microcrystalline cellulose is Celphere™ (Asahi Kasei Corporation, Japan).

### Silodosin

“Silodosin” is the compound 1-(3-hydroxypropyl)-5-[2-[[2-[2-(2,2,2-trifluoroethoxy)phenoxy]ethyl]amino]propyl]indoline-7-carboxamide (C₂₅H₃₂F₃N₃O₄, MW 495.53 g/mol).

According to some preferred embodiments, silodosin is in the form of its (R)-stereoisomer, namely, “(R)-silodosin”. (R)-silodosin is formally known as (−)-(R)-1-(3-hydroxypropyl)-5-[2-[[2-[2-(2,2,2-trifluoroethoxy)phenoxy]ethyl]amino]propyl]indoline-7-carboxamide. (R)-silodosin has the following formula:

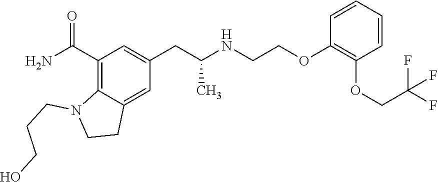 [SMILES: C[C@H](Cc1cc2c(c(C(N)=O)c1)N(CCCO)CC2)NCCOc1ccccc1OCC(F)(F)F]

(R)-silodosin is a powder that appears white or pale yellow/white (melting point ranging from about 105 to 109° C.). It is very soluble in acetic acid, freely soluble in alcohol, and very slightly soluble in water.

All references to “silodosin” include references to salts, solvates, multi-component complexes and/or liquid crystals thereof. All references to “silodosin” include references to polymorphs and/or crystal habits thereof. All references to “silodosin” include references to pharmaceutically acceptable prodrugs thereof. All references to “silodosin” include references to isotopically-labelled silodosin, including deuterated silodosin.

Silodosin can be in the form of pharmaceutically acceptable salts. Pharmaceutically acceptable salts of silodosin include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminium, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts.

Silodosin can be in polymorphic or amorphous form. According to some embodiments, silodosin is polymorphic. According to some embodiments, silodosin is amorphous.

Silodosin can be in the form of pharmaceutically acceptable solvates. Pharmaceutically acceptable solvates of silodosin include hydrates thereof.

### Binder

According to some embodiments, the binder is selected from cellulose polymers. In some embodiments, the binder is selected from hydroxypropyl cellulose (HPC), hydroxypropylmethylcellulose (HPMC), carboxymethylcellulose, methylcellulose, ethyl cellulose, povidone, polyvinylpyrrolidone, and mixtures thereof. In some preferred embodiments, the binder is hydroxypropyl cellulose (HPC).

### Antioxidant

According to some embodiments, the pellet further comprises at least one antioxidant. In some preferred embodiments, the drug layer comprises at least one antioxidant.

In some embodiments, the antioxidant is selected from phenols, vitamin E and derivatives thereof, vitamin C and derivatives thereof (e.g., ascorbic acid), citric acid, propanoic acid (propionic acid), erythorbic acid (or “D-erythro-hex-2-enosono-1,4-lactone”), fumaric acid (or “(2E)-but-2-enedioic acid”), malic acid (or potassium metabisulfite, sodium “2-hydroxybutanedioic acid”) methionine, metabisulfite, propyl gallate, sodium ascorbate, sodium thiosulphate, polyethylene glycol succinate (PGS), and mixtures thereof. In some embodiments, the vitamin E derivative is selected from d-alpha tocopherol, dl-alpha tocopherol, d-alpha tocopheryl acetate, dl-alpha tocopheryl acetate, d-alpha tocopheryl acid succinate, dl-alpha tocopheryl acid succinate, beta tocopherol, delta tocopherol, gamma tocopherol, vitamin E polyethylene glycol succinate (tocophersolan) (also named “Vitamin E TPGS”), and mixtures thereof. In some embodiments, the vitamin C derivative is selected from ascorbic acid, ascorbyl palmitate, erythorbic acid, sodium ascorbate, and mixtures thereof.

In some embodiments, the antioxidant is selected from phenols, vitamin E and derivatives thereof, vitamin C and derivatives thereof, propyl gallate, and mixtures thereof. In some embodiments, the antioxidant is selected from butylated hydroxytoluene (BHT), butylated hydroxyanisole (BHA), alpha-tocopherol, ascorbyl palmitate, propyl gallate, and mixtures thereof.

In some embodiments, the antioxidant is selected from phenols. In some embodiments, the antioxidant is selected from butylated hydroxytoluene (BHT), butylated hydroxyanisole (BHA), and mixtures thereof. In some preferred embodiments, the antioxidant is butylated hydroxytoluene (BHT).

In some embodiments, the antioxidant is selected from vitamin E and derivatives thereof. In some preferred embodiments, the antioxidant is alpha-tocopherol.

In some embodiments, the antioxidant is selected from vitamin C and derivatives thereof. In some embodiments, the antioxidant is ascorbyl palmitate.

Anti-Tacking and/or Charge Agent

According to some embodiments, the pellet further comprises at least one anti-tacking and/or charge agent. In some preferred embodiments, the drug layer comprises at least one anti-tacking and/or charge agent. In some preferred embodiments, the extended-release coating comprises at least one anti-tacking and/or charge agent.

In some embodiments, the anti-tacking and/or charge agent is selected from calcium carbonate (CaCO₃), talc, calcium phosphate, tribasic calcium silicate, colloidal silicon dioxide, hydrophobic colloidal silica, magnesium oxide, magnesium silicate, magnesium trisilicate, and mixtures thereof.

In some embodiments, the anti-tacking and/or charge agent is selected from inorganic carbonates, magnesium silicates, and mixtures thereof. In some embodiments, the anti-tacking and/or charge agent is selected from calcium carbonate (CaCO₃), talc, and mixtures thereof.

In some embodiments, the anti-tacking and/or charge agent is selected from inorganic carbonates. In some embodiments, the anti-tacking and/or charge agent is calcium carbonate (CaCO₃). In some preferred embodiments, the drug layer comprises calcium carbonate (CaCO₃).

In some embodiments, the anti-tacking and/or charge agent is selected from magnesium silicates. In some embodiments, the anti-tacking and/or charge agent is talc. In some preferred embodiments, the extended-release coating comprises talc.

### Plasticizer

According to some embodiments, the pellet further comprises at least one plasticizer. In some preferred embodiments, the extended-release coating comprises at least one plasticizer. The pellet can further comprise an enteric coating as described herein after and, in some preferred embodiments, this optional enteric coating comprises at least one plasticizer.

In some embodiments, the plasticizer is selected from citric acid esters. In some preferred embodiments, the plasticizer is triethyl citrate (TEC).

In some embodiments, the plasticizer is selected from diols. In some embodiments, the plasticizer is 1,2-propylene glycol.

### Sealing Coating

According to some preferred embodiments, the pellet further comprises: at least one sealing coating (c) surrounding the drug layer. Then, the extended-release coating surrounds the optional sealing coating instead of the drug layer.

The Applicant unexpectedly observed that, in silodosin ER compositions, migration of silodosin into the ER coating can sometimes be observed. The Applicant surprisingly found out that including an optional sealing coating can significantly prevent the migration of silodosin into the ER coating (especially with ethylcellulose ER coatings, e.g., Aquacoat® ECD). Moreover, the Applicant surprisingly found out that including a sealing coating can significantly prevent the interaction of silodosin with the ER coating (especially with vinyl polymers ER coatings, e.g., Kollicoat® SR 30 D).

In some embodiments, the optional sealing coating comprises at least one cellulose polymer. In some embodiments, the cellulose polymer is selected from hydroxypropyl methylcellulose (HPMC), carboxymethylcellulose, methylcellulose, ethyl cellulose, and mixtures thereof.

In some preferred embodiments, the optional sealing coating comprises hydroxypropyl methylcellulose (HPMC). In some preferred embodiments, the optional sealing coating is obtained by application of the coating agent Opadry® (Colorcon, US).

### Extended-Release Coating

In the pellets described herein, the sealing coating (when present) and the extended-release coating are distinct coatings. In other words, the same coating cannot be at the same time the “sealing coating” and the “extended-release coating”, as used herein.

According to some embodiments, the extended-release coating comprises at least one polyvinyl ester polymer. In some embodiments, the extended-release coating comprises at least one polyvinyl acetate polymer. In some preferred embodiments, the extended-release coating comprises polyvinyl acetate (PVA), i.e., PVA as a homopolymer. In some preferred embodiments, the extended-release coating comprises polyvinylpyrrolidone (PVP) (also commonly called “polyvidone” or “povidone”). In some embodiments, the extended-release coating comprises a mixture of vinyl polymers. In some preferred embodiments, extended-release coating comprises polyvinyl acetate (PVA) and polyvinylpyrrolidone (PVP).

According to some embodiments, the extended-release coating comprises at least one additive commonly included in coating agents. In some embodiments, the extended-release coating comprises at least one dispersing agent and/or at least one surfactant. Typically, dispersing agents and/or surfactants can be present in polymer coating agents, for example in order to stabilize the dispersion before application of the coating during the manufacturing process. In some embodiments, the extended-release coating comprises at least one dispersing agent. The dispersing agent can for example comprise povidone (PVP). In some embodiments, the extended-release coating comprises at least one surfactant. The surfactant can for example comprise sodium lauryl sulfate (SLS).

In some embodiments, the extended-release coating comprises from about 70% to 99% w/w of the polyvinyl acetate (PVA) polymer or copolymer, in weight by weight of the extended-release coating. In some embodiments, the extended-release coating comprises from about 75% to 99% w/w, preferably from about 80% to 98% w/w, more preferably from about 85% to 95% w/w, of the polyvinyl acetate (PVA) polymer or copolymer, in weight by weight of the extended-release coating. In some embodiments, the extended-release coating comprises at least about 70% w/w, at least about 75% w/w, at least about 80% w/w, at least about 85% w/w, or at least about 90% w/w, of the polyvinyl acetate (PVA) polymer or copolymer, in weight by weight of the extended-release coating. In some preferred embodiments, the extended-release coating comprises about 90% w/w of the polyvinyl acetate (PVA) polymer or copolymer, in weight by weight of the extended-release coating.

In some embodiments, the extended-release coating comprises from about 6% to 15% w/w of polyvinylpyrrolidone (PVP), in weight by weight of the extended-release coating. In some embodiments, the extended-release coating comprises from about 6% to 15% w/w, preferably from about 7% to 13% w/w, more preferably from about 8% to 11% w/w, of polyvinylpyrrolidone (PVP), in weight by weight of the extended-release coating. In some embodiments, the extended-release coating comprises about 15% w/w or less, about 14% w/w or less, about 13% w/w or less, about 12% w/w or less, about 11% w/w or less, about 10% w/w or less, or about 9% w/w or less, of polyvinylpyrrolidone (PVP), in weight by weight of the extended-release coating. In some preferred embodiments, the extended-release coating comprises about 9% w/w of polyvinylpyrrolidone (PVP), in weight by weight of the extended-release coating.

In some preferred embodiments, the extended-release coating comprises sodium lauryl sulfate (SLS). In some embodiments, the extended-release coating comprises from about 0.6% to 1.5% w/w of sodium lauryl sulfate (SLS), in weight by weight of the extended-release coating. In some embodiments, the extended-release coating comprises from about 0.6% to 1.5% w/w, preferably from about 0.7% to 1.3% w/w, more preferably from about 0.8% to 1.1% w/w, of sodium lauryl sulfate (SLS), in weight by weight of the extended-release coating. In some embodiments, the extended-release coating comprises about 1.5% w/w or less, about 1.4% w/w or less, about 1.3% w/w or less, about 1.2% w/w or less, about 1.1% w/w or less, about 1% w/w or less, of sodium lauryl sulfate (SLS), in weight by weight of the extended-release coating. In some preferred embodiments, the extended-release coating comprises about 1% w/w of sodium lauryl sulfate (SLS), in weight by weight of the extended-release coating.

In some preferred embodiments, the extended-release coating is obtained by application of the coating agent “Kollicoat® SR 30 D” (BASF Pharma, Germany), in short “KSR”. Kollicoat® SR 30 D is an aqueous dispersion with a solids content of 30%. The dispersion consists of 27% polyvinyl acetate (PVA), about 2.7% povidone (PVP), about 0.3% sodium lauryl sulfate (SLS), and water.

The Applicant unexpectedly observed that, in silodosin compositions wherein the ER coating is ethylcellulose (such as, for example, Aquacoat® ECD), the release profile of silodosin is unstable. Unstable release patterns can possibly render the continuous contraceptive effect instable and thus unreliable. The Applicant carried out significant research and tested a wide range of common ER polymers, none of which led to an appropriate and/or sufficiently stable silodosin release profile. In the end, the Applicant surprisingly found that using vinyl polymers (such as, for example, Kollicoat® SR 30 D) as ER coating significantly improved the stability of the release profile of silodosin.

However, the Applicant also surprisingly found that the release profile of the silodosin pellets described herein coated with ER vinyl polymers (such as, for example, Kollicoat® SR 30 D) were strongly “pH-dependent” in the sense that the release of silodosin was accelerated by acidic pH of the environment medium. In the art, this property is generally considered disadvantageous for a therapeutic or contraceptive formulation, because this kind of “pH-dependent” composition typically starts to dissolve in the stomach (wherein the pH is very acid) in conditions that cannot be properly controlled and can also depend on the particulars of the subject. Therefore, a skilled person would have a strong expectation that this kind of “pH-dependent” composition would cause an uncontrolled “burst release” (or “burst release effect”) when going through the stomach, thereby limiting or suppressing the ER effect.

Moreover, the Applicant also surprisingly found that the silodosin pellets described herein coated with ER vinyl polymers (such as, for example, Kollicoat® SR 30 D) presented a “lag-time” during in vitro assays, i.e., the release of silodosin did not start immediately, but was instead delayed for some time (for example, for about 1 h). Thus, these pellets were “delayed-release” compositions in the sense of the present disclosure. In the art, this property is generally considered disadvantageous for a contraceptive formulation, because it may be desired that the contraceptive effect starts as soon as possible after the administration. However, when a continuous contraception is required, the contraceptive composition is generally taken daily, so that a limited delay in silodosin release should not affect the reliability of the contraception. Therefore, a “delayed-release” may be convenient for contraception purposes, as long as the “lag time” remains relatively short compared with the overall duration of contraceptive effect.

As explained hereinabove, as the silodosin pellets coated with ER vinyl polymers (such as, for example, Kollicoat® SR 30 D) were “pH-dependent”, there was a significant risk of “burst release effect” when they are administered to subjects, so that the ER release of silodosin observed in vitro would not be maintained in vivo. However, the Applicant surprisingly found that no burst release effect whatsoever was observed in the treated subjects, which was fully unexpected in view of the general knowledge in the art. Without wishing to be bound by theory, the Applicant believes that some association may exist between the absence of in vivo “burst release effect” and the “lag time” observed in vitro, which could explain the surprising technical effect of the pellets and dosage forms described herein.

### Enteric Coating

According to some embodiments, the pellet further comprises: at least one enteric coating (e). In some preferred embodiments, the enteric coating is surrounding said extended-release coating. In some other embodiments, the enteric coating is surrounded by the extended-release coating and surrounding the drug layer or the optional sealing coating. In some preferred embodiments, the drug layer or the optional sealing coating is coated by at least one extended-release coating, then by at least one enteric coating. In some other embodiments, the drug layer or the optional sealing coating is coated by at least one enteric coating, then by at least one extended-release coating.

In the pellets described herein, the sealing coating (when present) and the enteric coating are distinct coatings. In other words, the same coating cannot be at the same time the “sealing coating” and the “enteric coating”, as used in the present application.

In the pellets described herein, the extended-release coating and the enteric coating are distinct coatings. In other words, the same coating cannot be at the same time the “extended-release coating” and the “enteric coating”, as used in the present application.

In some embodiments, the enteric coating comprises at least one polymer selected from acrylate polymers, cellulose polymers, and mixtures thereof.

In some embodiments, the enteric coating comprises at least one acrylate polymer.

In some preferred embodiments, the enteric coating comprises at least one acrylate copolymer. In some embodiments, the enteric coating comprises at least one methacrylic acid ethylacrylate (MAE) copolymer. In some preferred embodiments, the enteric coating comprises a methacrylic acid ethylacrylate copolymer (1:1) (“MAE 1:1”).

In some embodiments, the enteric coating comprises at least one polymethacrylate polymer (e.g., Eudragit® NE).

In some embodiments, the enteric coating comprises from about 70% to 99% w/w of the acrylate copolymer(s), in weight by weight of the enteric coating. In some preferred embodiments, the enteric coating comprises about 97% w/w of the acrylate copolymer(s), in weight by weight of the enteric coating.

In some embodiments, the enteric coating comprises at least one polyethoxylated sorbitan fatty ester. In some embodiments, the enteric coating comprises at least one polysorbate. In some preferred embodiments, the enteric coating comprises polyoxyethylene (20) sorbitan monooleate (“polysorbate 80”).

In some embodiments, the enteric coating comprises from about 1.5% to 3% w/w of the polyethoxylated sorbitan fatty ester(s), in weight by weight of the enteric coating. In some preferred embodiments, the enteric coating comprises about 2.3% w/w of the polyethoxylated sorbitan fatty ester(s), in weight by weight of the enteric coating.

In some embodiments, the enteric coating comprises a mixture of at least one acrylate polymer and at least one polyethoxylated sorbitan fatty ester. In some preferred embodiments, the enteric coating comprises a mixture of methacrylic acid ethylacrylate (MAE) copolymer and polysorbate 80.

In some preferred embodiments, the enteric coating is obtained by application of the coating agent “Kollicoat® MAE 30 DP” (BASF Pharma, Germany), in short “KMAE”. Kollicoat® MAE 30 DP is an aqueous dispersion with a solids content of 30% (w/w). Based on the solid content, the dispersion consists of 97% (w/w) methacrylic acid and ethylacrylate copolymer (1:1), about 2.3% (w/w) polysorbate 80 and about 0.7% (w/w) sodium lauryl sulfate.

In some embodiments, the enteric coating comprises at least one cellulose polymer. In some embodiments, the enteric coating comprises at least one carboxymethylcellulose polymer. In some embodiments, the cellulose polymer is selected from Hypromellose Acetate Succinate (e.g., AQOAT®), cellulose acetate phthalate (CAP), Hypromellose Phthalate (HPMPC), and mixtures thereof.

In some embodiments, the enteric coating comprises at least one poly(methyl vinyl ether/maleic anhydride) copolymer (e.g., Gantrez™).

In some embodiments, the enteric coating comprises at least one polyvinyl acetate phthalate (e.g., “Opadry® enteric”).

The Applicant surprisingly found that adding an enteric coating (such as, for example, Kollicoat® MAE 30DP) renders the pellet more resistant to acidity, for example non-significant release of silodosin in a simulated gastric fluid for 2 h.

### Exemplary Pellets

According to some embodiments, the drug layer comprises silodosin or a pharmaceutically acceptable salt and/or solvate thereof, at least one binder, at least one antioxidant, and at least one anti-tacking and/or charge agent.

The Applicant unexpectedly observed that, when manufacturing the drug layer of the silodosin compositions at laboratory scale (small-scale experiments), it was advantageous to include a minimal amount of a binder and a minimal amount of anti-tacking and/or charge agent in order to improve the coating formation, but their presence was identified as a possible cause of degradation of the silodosin. The Applicant surprisingly found that including at least one antioxidant in the drug layer significantly prevented the degradation of the silodosin during the manufacturing process.

According to some embodiments, the pellet comprises a pellet comprising or consisting essentially of the inert core surrounded by a drug layer (herein referred to as “pellet A”). Pellet A is useful, in particular, as an intermediate in the manufacture of another pellet. Pellet A can also be useful, in particular, as an immediate release (IR) silodosin formulation.

According to some embodiments, the pellet comprises a pellet comprising or consisting essentially of the inert core surrounded by a drug layer, the drug layer being surrounded by a sealing coating (herein referred to as “pellet B”). Pellet B is useful, in particular, as an intermediate in the manufacture of another pellet. Pellet B can also be useful, in particular, as an immediate release (IR) silodosin formulation.

According to some embodiments, the pellet comprises a pellet comprising or consisting essentially of the inert core surrounded by a drug layer, the drug layer being optionally surrounded by a sealing coating, the drug layer or the optional sealing coating being surrounded by an extended-release coating (herein referred to as “pellet C”). Pellet C is useful, in particular, as part of a contraceptive composition.

According to some embodiments, the pellet comprises a pellet comprising or consisting essentially of the inert core surrounded by a drug layer, the drug layer being optionally surrounded by a sealing coating, the drug layer or the optional sealing coating being surrounded by an extended-release coating, the extended-release coating being surrounded by an enteric coating (herein referred to as “pellet D”). Pellet D is useful, in particular, as part of a contraceptive composition.

According to some embodiments, the pellet comprises a pellet comprising or consisting essentially of the inert core surrounded by a drug layer, the drug layer being optionally surrounded by a sealing coating, the drug layer or the optional sealing coating being surrounded by an enteric coating (herein referred to as “pellet E”). Pellet E is useful, in particular, as an intermediate in the manufacture of another pellet. Pellet E can also be useful, in particular, as a delayed and immediate release silodosin formulation.

According to some embodiments, the pellet comprises a pellet comprising or consisting essentially of the inert core surrounded by a drug layer, the drug layer being optionally surrounded by a sealing coating, the drug layer or the optional sealing coating being surrounded by an enteric coating, the enteric coating being surrounded by an extended-release coating (herein referred to as “pellet F”). Pellet F is useful, in particular, as part of a contraceptive composition.

The Applicant surprisingly found that, when formulating silodosin in the form of pellet C, pellet D, or pellet F as described herein, the determination or the adjustment of the optimal dose of silodosin (“dose ranging”) was easier than with other ER compositions.

The Applicant surprisingly found that, when formulating silodosin in the form of pellet C, pellet D, or pellet F as described herein, the determination or the adjustment of the release rate of silodosin was easier than with other ER compositions.

### Exemplary Formulations

According to some embodiments, the pellet comprises:
(a) an inert core comprising cellulose microspheres;
(b) at least one drug layer applied to the inert core, wherein the drug layer comprises: silodosin, hydroxypropyl cellulose (HPC), calcium carbonate (CaCO₃), and butylated hydroxytoluene (BHT);
(c) at least one sealing coating surrounding the drug layer, wherein the sealing coating comprises hydroxypropyl methylcellulose (HPMC); and
(d) at least one extended-release coating surrounding the drug layer or the optional sealing coating, wherein the extended-release coating comprises: polyvinyl acetate (PVA), polyvinylpyrrolidone (PVP), triethyl citrate (TEC), and talc.

In some embodiments, the pellet further comprises:
(e) at least one enteric coating, wherein the enteric coating is either surrounding the extended-release coating, or surrounded by the extended-release coating and surrounding the optional sealing coating or the drug layer, wherein the enteric coating comprises: a methacrylic acid ethylacrylate copolymer 1:1 (MAE 1:1), and triethyl citrate (TEC).

### Amounts of Ingredients

According to some embodiments, the pellet comprises a pellet A comprising or consisting essentially of:
from about 24 to 95% w/w, preferably 43.3 to 91.5% w/w, more preferably 74 to 83.5% w/w, of the inert core,
from about 5 to 76% w/w, preferably 8.5 to 56.7% w/w, more preferably 16.5 to 26% w/w, of the drug layer, the drug layer comprising:
   from about 5 to 25% w/w, preferably 8 to 18% w/w, more preferably 11 to 15% w/w, of the silodosin,
   from about 0.1 to 7.5% w/w, preferably 0.4 to 3.6% w/w, more preferably 0.88 to 1.8% w/w of the binder,
   from about 0 to 25% w/w, preferably 0.01 to 10% w/w, more preferably 0.022 to 0.09%, of at least one antioxidant, and
   from about 0 to 25% w/w, preferably 2 to 13.5% w/w, more preferably 4.4 to 9% w/w, of at least one anti-tacking and/or charge agent,
in weight relative to the total weight of the pellet A.

According to some embodiments, the pellet comprises a pellet B comprising or consisting essentially of:
from about 90 to 100% w/w, preferably 92.5 to 97.5% w/w, more preferably 94 to 96% w/w, of the pellet A, and
from about 0 to 10% w/w, preferably 2.5 to 7.5% w/w, more preferably 4 to 6% w/w, of at least one sealing coating,
in weight relative to the total weight of the pellet B.

According to some embodiments, the pellet comprises a pellet C comprising or consisting essentially of:
from about 50 to 98% w/w, preferably 75 to 96.5% w/w, more preferably 82 to 94% w/w, of the pellet A or the pellet B, and
an extended-release coating comprising:
   from about 0.5 to 47% w/w, preferably 1.6 to 19.1% w/w, more preferably 4.7 to 12.25% w/w, of at least one extended-release agent,
   from about 0.02 to 0.7% w/w, preferably 0.06 to 1.6% w/w, more preferably 0.19 to 0.75% w/w, of at least one plasticizer, and
   from about 0.3 to 23% w/w, preferably 0.3 to 8.6% w/w, more preferably 1.3 to 5.15% w/w, of at least one anti-tacking agent,
in weight relative to the total weight of the pellet C.

According to some embodiments, the pellet comprises a pellet D comprising or consisting essentially of:
from about 50 to 95% w/w, preferably 62 to 88% w/w, more preferably 70 to 80% w/w, of the pellet C, and
an enteric coating comprising:
   from about 4 to 49.5% w/w, preferably 10 to 36%, more preferably 17.5 to 27.5% w/w, of at least one enteric agent, and
   from about 0.0.04 to 10% w/w, preferably 0.5 to 5.5% w/w, more preferably 1.4 to 3.3% w/w, of at least one plasticizer,
in weight relative to the total weight of the pellet D.

According to some embodiments, the pellet comprises:

a pellet A comprising or consisting essentially of:
from about 24 to 95% w/w, preferably 43.3 to 91.5% w/w, more preferably 74 to 83.5% w/w, of the inert core,
from about 5 to 76% w/w, preferably 8.5 to 56.7% w/w, more preferably 16.5 to 26% w/w, of the drug layer, the drug layer comprising:
   from about 5 to 25% w/w, preferably 8 to 18% w/w, more preferably 11 to 15% w/w, of the silodosin,
   from about 0.1 to 7.5% w/w, preferably 0.4 to 3.6% w/w, more preferably 0.88 to 1.8% w/w, of the binder,
   from about 0 to 20% w/w, preferably 0.01 to 10% w/w, more preferably 0.022 to 0.09%, of at least one antioxidant, and
   from about 0 to 25% w/w, preferably 2 to 13.5% w/w, more preferably 4.4 to 9% w/w, of at least one anti-tacking and/or charge agent,
in weight relative to the total weight of the pellet A;
wherein the pellet A is optionally comprised in a pellet B comprising or consisting essentially of:
from about 90 to 100% w/w, preferably 92.5 to 97.5% w/w, more preferably 94 to 96% w/w, of the pellet A, and
from about 0 to 10% w/w, preferably 2.5 to 7.5% w/w, more preferably 4 to 6% w/w, of at least one sealing coating surrounding the drug layer, in weight relative to the total weight of the pellet B;
wherein the pellet A or the pellet B is comprised in a pellet C comprising or consisting essentially of:
from about 50 to 98% w/w, preferably 75 to 96.5% w/w, more preferably 82 to 94% w/w of the pellet A or the pellet B, and
an extended-release coating comprising:
   from about 0.5 to 47% w/w, preferably 1.6 to 19.1% w/w, more preferably 4.7 to 12.25% w/w, of at least one extended-release agent,
   from about 0.02 to 0.7% w/w, preferably 0.06 to 1.6% w/w, more preferably 0.19 to 0.75% w/w of at least one plasticizer, and
   from about 0.3 to 23% w/w, preferably 0.3 to 8.6% w/w, more preferably 1.3 to 5.15% w/w of at least one anti-tacking agent;
in weight relative to the total weight of the pellet C; and
wherein the pellet C is optionally comprised in a pellet D comprising or consisting essentially of:
from about 50 to 95% w/w, preferably 62 to 88% w/w, more preferably 70 to 80% w/w, of the pellet C, and
an enteric coating comprising:
   from about 4 to 49.5% w/w, preferably 10 to 36%, more preferably 17.5 to 27.5% w/w, of at least one enteric agent, and
   from about 0.04 to 10% w/w, preferably 0.5 to 5.5% w/w, more preferably 1.4 to 3.3% w/w, of at least one plasticizer,
in weight relative to the total weight of the optional pellet D.

In some embodiments, the pellet A or the pellet B is comprised in a pellet C comprising or consisting essentially of:
from about 75 to 93% w/w, preferably 82 to 88% w/w, of the pellet A or the pellet B; and
an extended-release coating comprising:
   from about 11.25 to 119.1% w/w, preferably 6.25 to 12.25% w/w, of at least one extended-release agent;
   from about 0.4 to 1.6% w/w, preferably 0.25 to 0.75% w/w, of at least one plasticizer; and
   from about 2.25 to 8.6% w/w, preferably 1.75 to 5.15% w/w, of at least one anti-tacking agent;
in weight relative to the total weight of the pellet C.

In some embodiments, the pellet A or the pellet B is comprised in a pellet C comprising or consisting essentially of:
from about 80 to 96.5% w/w, preferably 91 to 94% w/w, of the pellet A or the pellet B; and
an extended-release coating comprising:
   from about 1.6 to 15.3% w/w, preferably 4.7 to 6.1% w/w, of at least one extended-release agent;
   from about 0.06 to 1.5% w/w, preferably 0.19 to 0.37% w/w, of at least one plasticizer; and
   from about 0.3 to 6.9% w/w, preferably 1.3 to 2.6% w/w, of at least one anti-tacking agent;
in weight relative to the total weight of the pellet C.

According to some embodiments, the pellet comprises a pellet E comprising or consisting essentially of:
from about 50 to 95% w/w of a pellet A as described herein or a pellet B as described herein, and
an enteric coating comprising:
   from about 4 to 49.5% w/w, preferably 10 to 36%, more preferably 17.5 to 27.5% w/w, of at least one enteric agent, and
   from about 0.04 to 10% w/w, preferably 0.5 to 5.5% w/w, more preferably 1.4 to 3.3% w/w, of at least one plasticizer,
in weight relative to the total weight of the pellet E.

According to some embodiments, the pellet comprises a pellet F comprising or consisting essentially of:
from about 50 to 98% w/w, preferably 75 to 96.5% w/w, more preferably 82 to 94% w/w, of the pellet E, and
an extended-release coating comprising:
   from about 0.5 to 47% w/w, preferably 1.6 to 19.1% w/w, more preferably 4.7 to 12.25% w/w, of at least one extended-release agent,
   from about 0.02 to 0.7% w/w, preferably 0.06 to 1.6% w/w, more preferably 0.19 to 0.75% w/w of at least one plasticizer, and
   from about 0.3 to 23% w/w, preferably 0.3 to 8.6% w/w, more preferably 1.3 to 5.15% w/w, of at least one anti-tacking agent,
in weight relative to the total weight of the pellet F.

According to some embodiments, the pellet comprises:

a pellet A comprising or consisting essentially of:
from about 24 to 95% w/w, preferably 43.3 to 91.5% w/w, more preferably 74 to 83.5% w/w, of the inert core,
from about 5 to 76% w/w, preferably 8.5 to 56.7% w/w, more preferably 16.5 to 26% w/w, of the drug layer, the drug layer comprising:
   from about 5 to 25% w/w, preferably 8 to 18% w/w, more preferably 11 to 15% w/w, of the silodosin,
   from about 0.1 to 7.5% w/w, preferably 0.4 to 3.6% w/w, more preferably 0.88 to 1.8% w/w, of the binder,
   from about 0 to 20% w/w, preferably 0.01 to 10% w/w, more preferably 0.022 to 0.09%, of at least one antioxidant, and
   from about 0 to 25% w/w, preferably 2-13.5% w/w, more preferably 4.4 to 9% w/w, of at least one anti-tacking and/or charge agent,
in weight relative to the total weight of the pellet A;
wherein the pellet A is optionally comprised in a pellet B comprising or consisting essentially of:
from about 90 to 100% w/w, preferably 92.5 to 97.5% w/w, more preferably 94 to 96% w/w, of the pellet A, and
from about 0 to 10% w/w, preferably 2.5 to 7.5% w/w, more preferably 4 to 6% w/w, of at least one sealing coating surrounding the drug layer, in weight relative to the total weight of the pellet B;
wherein the pellet A or the pellet B is comprised in a pellet E comprising or consisting essentially of:
from about 50 to 95% w/w of the pellet A or the pellet B, and
an enteric coating comprising:
   from about 4 to 49.5% w/w, preferably 10 to 36%, more preferably 17.5 to 27.5% w/w, of at least one enteric agent, and
   from about 0.0.04 to 10% w/w, preferably 0.5 to 5.5% w/w, more preferably 1.4 to 3.3% w/w, of at least one plasticizer,
in weight relative to the total weight of the pellet E.
wherein the pellet E is comprised in a pellet F comprising or consisting essentially of:
from about 50 to 98% w/w, preferably 75 to 96.5% w/w, more preferably 82 to 94% w/w, of the pellet E,
an extended-release coating comprising:
   from about 0.5 to 47% w/w, preferably 1.6 to 19.1% w/w, more preferably 4.7 to 12.25% w/w, of at least one extended-release agent,
   from about 0.02 to 0.7% w/w, preferably 0.06 to 1.6% w/w, more preferably 0.19 to 0.75% w/w, of at least one plasticizer, and
   from about 0.3 to 23% w/w, preferably 0.3 to 8.6% w/w, more preferably 1.3 to 5.15% w/w of, at least one anti-tacking agent,
in weight relative to the total weight of the pellet F.

### Weight Gain

“Weight gain” refers to the increase in weight in a final object that results from a step of applying at least one coating agent (e.g., a polymer) onto a starting object (e.g., an inert core or a pellet), thereby obtaining the final object wherein the staring object is coated by at least one coating material. The weight gain is a parameter commonly used in the field of formulation to characterize the thickness of a coating, because direct measurement of coating thickness requires complicated and costly methods, whereas the gain can be simply estimated by weighing using common material, such as for example laboratory scale. The weight gain WGₙ (%) after “n” coating agents have been applied to the starting object (“n” being an integer higher than 0) is calculated as follows: WGₙ=100*(Wₙ)/W₀, wherein W₀ represents the weight of the starting object and Wₙ represents the dry weight of the coating agent, Wₙ and W₀ being expressed in the same unit (e.g., in gram or “g”). In the present application, unless otherwise indicated, the weight gain is expressed when only one coating has been applied, i.e., n is 1. For example, a recitation such as “said [Coating agent 1] is applied onto the [Pellet 0] at a weight gain of 10%” means that W₀ is the weight of “Pellet 0” (the starting object), W₁ is the dry weight of Coating agent 1 and 100*(W₁)/W₀=10%.

According to some embodiments, the drug layer is applied onto the inert core at a weight gain ranging from about 5 to 318%, preferably 10 to 105%, more preferably 19 to 35%.

According to some embodiments, the optional sealing coating agent is applied onto the drug layer at a weight gain ranging from 0 to about 11%, preferably 2.5 to 8%, more preferably 13.5 to 22%.

According to some embodiments, the extended-release coating agent is applied onto the drug layer or the optional sealing coating at a weight gain ranging from about 2 to 100%, preferably 3.5 to 33%, more preferably 6 to 22%.

According to some embodiments, the optional enteric coating agent is applied onto the extended-release coating at a weight gain ranging from 0 to about 100%, preferably 13.5 to 61%, more preferably 25 to 43%.

According to some embodiments, the pellet comprises:
(a) the inert core,
(b) the drug layer applied onto the inert core at a weight gain ranging from about to 318%, preferably 10 to 105%, more preferably 19 to 35%;
(c) the optional sealing coating agent applied onto the drug layer at a weight gain ranging from 0 to about 11%, preferably 2.5 to 8%, more preferably 13.5 to 22%;
(d) the extended-release coating agent applied onto the drug layer or the optional sealing coating at a weight gain ranging from about 2 to 100%, preferably 3.5 to 33%, more preferably 6 to 22%; and/or
(e) the optional enteric coating agent applied onto the extended-release coating at a weight gain ranging from 0 to about 100%, preferably 13.5 to 61%, more preferably 25 to 43%.

According to some embodiments, the enteric coating agent is applied onto the drug layer or the optional sealing coating at a weight gain ranging from 0.1 to about 100%.

According to some embodiments, the extended-release coating agent is applied onto the enteric coating at a weight gain ranging from about 2 to 100%.

According to some embodiments, the pellet comprises:
(a) the inert core,
(b) the drug layer applied onto the inert core at a weight gain ranging from about to 318%, preferably 10 to 105%, more preferably 19 to 35%;
(c) the optional sealing coating agent applied onto the drug layer at a weight gain ranging from 0 to about 11%, preferably 2.5 to 8%, more preferably 13.5 to 22%;
(d) the enteric coating agent applied onto the drug layer or the optional sealing coating at a weight gain ranging from 0.1 to about 100%; and/or
(e) the extended-release coating agent applied onto the enteric coating at a weight gain ranging from about 2 to 100%.

### Plurality of Pellets

An object of the technology described herein is a plurality of pellets as described herein.

According to some embodiments, at least one pellet is selected from pellet A, pellet B, pellet C, pellet D, pellet E, and pellet F as described herein. In some embodiments, each pellet in the plurality has the same structure, i.e., each pellet is pellet A, each pellet is pellet B, each pellet is pellet C, each pellet is pellet D, each pellet is pellet E, or each pellet is pellet F. In some embodiments, the plurality comprises at least two types of pellets selected from pellet A, pellet B, pellet C, pellet D, pellet E, and pellet F. In some embodiments, the plurality comprises at least one pellet selected from pellet A, pellet B, and pellet E; and at least one pellet selected from pellet C, pellet D, and pellet F. A plurality of pellets A, pellets B or pellets E is useful, in particular, as an intermediate in the manufacture of another plurality of pellets.

In some embodiments, at least one pellet is selected from pellet A, pellet B, and pellet E as described herein. In some embodiments, each pellet in the plurality has the same structure, i.e., each pellet is pellet A, each pellet is pellet B, or each pellet is pellet E. In some preferred embodiments, each pellet in the plurality is pellet A. In some preferred embodiments, each pellet in the plurality is pellet B. A plurality of pellets A, pellets B or pellets E is useful, in particular, as part of a contraceptive composition.

In some embodiments, at least one pellet is selected from pellet C, pellet D, and pellet F as described herein. In some embodiments, each pellet in the plurality has the same structure, i.e., each pellet is pellet C, each pellet is pellet D, or each pellet is pellet F. In some preferred embodiments, each pellet in the plurality is pellet C. In some preferred embodiments, each pellet in the plurality is pellet D. A plurality of pellets C, pellets D or pellets F is useful, in particular, as part of a contraceptive composition.

### Compositions

Another object of the technology described herein is a composition comprising at least one pellet as described herein.

According to some embodiments, the composition consists of a plurality of pellets as described herein. According to some embodiments, the composition consists of one pellet as described herein.

According to some preferred embodiments, the composition is a contraceptive composition.

According to some embodiments, the composition is a dosage form.

“Dosage form” refers to the form in which a dose of an active ingredient (e.g., an “effective amount” thereof) is to be administered to a subject. The active ingredient is generally administered as part of a formulation that includes non-medical agents (e.g., pharmaceutically acceptable carriers). The dosage form has unique physical and pharmaceutical characteristics. Dosage forms can comprise at least one pharmaceutical composition. Dosage forms can for example be solid, liquid or gaseous. “Dosage forms” can include, for example, a capsule (e.g., a hard-shell or soft-shell capsule such as, for example, a gel caplet [“gel-cap”]), a tablet, a caplet, a syrup, a liquid composition, a powder, a concentrated powder, a concentrated powder admixed with a liquid, a swallowable form, a granulated form, a pellet form, an oral liquid solution, as well as mixtures and/or combinations thereof. Dosage forms can also include at least one subdermal implant, transdermal patch, injectable form, nasal spray, adhesive tablet, or transmucosally delivered solution.

In some embodiments, the dosage form is a capsule. In some preferred embodiments, the capsule is a hard-shell capsule. In some embodiments, the capsule is a functional capsule. In some embodiments, the capsule is an enteric capsule.

“Functional capsule” refers to a capsule, typically a polymer comprising capsule, which comprise an oral medication and confer a particular feature to the dissolution profile of the said oral medication.

“Enteric capsule” refers to a capsule, typically a polymer comprising-capsule, which comprise an oral medication and prevents its dissolution or disintegration in the gastric environment. Enteric capsules are useful either in protecting drugs from the acidity of the stomach, protecting the stomach from the detrimental effects of the drug, or to release the drug after the stomach (usually in the upper tract of the intestine). Typically, an enteric capsule is expected to dissolves at a pH equal or above about 5.5.

According to some embodiments, the composition is a pharmaceutical composition.

According to some embodiments, the composition is a medicament.

According to some embodiments, the pellets are lubricated with at least one anti-tacking agent before they are filled into the dosage form. In some preferred embodiments, the anti-tacking agent is talc.

According to some embodiments, the composition comprises a plurality of pellets as described herein.

According to some embodiments, the composition comprises an amount of silodosin ranging from about 0.5 to 50 mg.

According to some embodiments, the composition comprises an amount of silodosin ranging from about 0.5 to 4 mg, preferably ranging from about 1 to 8 mg, more preferably ranging from about 2 to 12 mg, furthermore preferably ranging from about 4 to 16 mg. In some embodiments, the composition comprises an amount of silodosin ranging from about 1 to 8 mg. In some specific embodiments, the composition comprises an amount of silodosin ranging from about 2 to 12 mg. In some further specific embodiments, the composition comprises an amount of silodosin ranging from about 4 to 16 mg.

According to some embodiments, the composition comprises an amount of silodosin ranging from about 4 to 32 mg, preferably ranging from about 8 to 28 mg, more preferably ranging from about 12 to 24 mg, furthermore preferably ranging from about 16 to 20 mg. In some embodiments, the composition comprises an amount of silodosin ranging from about 8 to 28 mg. In some specific embodiments, the composition comprises an amount of silodosin ranging from about 12 to 24 mg. In some further specific embodiments, the composition comprises an amount of silodosin ranging from about 16 to 20 mg.

According to some embodiments, the composition comprises an amount of silodosin ranging from about 5 to 50 mg, preferably ranging from about 10 to 45 mg, more preferably ranging from about 15 to 40 mg, furthermore preferably ranging from about 20 to 35 mg. In some embodiments, the composition comprises an amount of silodosin ranging from about 10 to 45 mg. In some specific embodiments, the composition comprises an amount of silodosin ranging from about 15 to 40 mg. In some further specific embodiments, the composition comprises an amount of silodosin ranging from about 20 to 35 mg.

According to some embodiments, the composition comprises an amount of silodosin of about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

According to some preferred embodiments, the entirety of the silodosin that is present in the composition is comprised in the pellet or the plurality of pellets described herein. According to some other embodiments, part of the silodosin present in the composition is not comprised in the pellet or plurality of pellets described herein.

In some embodiments, the composition comprises from about 2.5 to 10 mg, preferably about 5 mg, of lubricated pellets (e.g., lubricated pellets C). In some embodiments, the composition comprises from about 125 to 500 mg, preferably about 250 mg of lubricated pellets (e.g., lubricated pellets C). In some embodiments, the composition comprises about 250 to 1000 mg, preferably about 500 mg of lubricated pellets (e.g., lubricated pellets C).

According to some embodiments, the composition further comprises at least another contraceptive agent, i.e., a contraceptive agent other than silodosin.

### Kit

Another object of the present disclosure is a kit-of-parts (in short “kit”) comprising a composition as described herein.

According to some embodiments, the kit comprises a manufacture such as, for example, a package or a container. According to some embodiments, the kit comprises instructions for use. The kit can be promoted, distributed, or sold as a unit for performing the methods or uses of the technology described herein.

### Methods and Uses

Another object of the technology described herein is a contraception method for a male subject comprising a step of administration of a composition as described herein to the male subject.

Another object of the technology described herein is the use of a composition as described herein in a contraception method for a male subject.

Another object of the technology described herein is a composition as described herein for use as a male contraceptive (i.e., a contraceptive for a male subject). Another object of the technology described herein is a composition as described herein for use in a contraception method for a male subject.

Another object of the technology described herein is the use of a composition as described herein, in the manufacture of a medicament for male contraception. Another object of the technology described herein is the use of a composition as described herein, in the manufacture of a medicament for a contraception method for a male subject.

### Contraceptive Methods

The silodosin-containing modified-release formulations described herein can be used in a method of male contraception comprising once daily oral administration of a dosage form comprising such a formulation. Such administration, e.g., at about the same time each day, induces or triggers a continuous, reversible aspermia, azoospermia, or severe oligozoospermia in the male subject, providing contraceptive effect. In one embodiment, after an initial period of doses on at least two consecutive days, the contraception is not impaired by a delay of the subsequent once daily intake. In one embodiment, the initial period of consecutive days is at least two consecutive days, with administration at about the same time each day. In another embodiment, the initial period of consecutive days is at least 5 days.

In another embodiment, after the initial period of doses on consecutive days, the intake of a next dose administration can be delayed from 6 to 18 hours after the last regular daily dose time, and the condition of aspermia, azoospermia or severe oligozoospermia is maintained in the male subject. In another embodiment, after the initial period of consecutive days, the continuous state of aspermia, azoospermia or severe oligozoospermia is not impaired by a delay of the subsequent once daily intake, supporting the contraception method for 26 hours to 48 hours, preferably 30 hours to 48 hours, more preferably 36 to 48 hours, as of the last intake time. According to the aforementioned embodiments, the delay of a subsequent daily administration does not exceed 2, 4, 6, 8, 10 12, 16, 20 or 24 hours from the day-before intake time.

In another embodiment, after an initial period of doses on at least two consecutive days, three consecutive days, four consecutive days, five consecutive days, six consecutive days, eight consecutive days or more, the administration of the daily dosing regimen does not necessarily occur at about the same time each day, but each daily dose is administered within twenty-four hours of the previous daily dose.

In one embodiment, the contraception method is carried on with once-daily dosing for at least eight days, at least 10 days, at least two weeks, at least three weeks, at least four weeks, at least one month, at least six weeks at least eight weeks at least two months, at least ten weeks, at least twelve weeks, at least three months, at least fourteen weeks, at least sixteen weeks, at least four months, at least eighteen weeks, at least twenty weeks, at least five months, at least twenty two weeks, at least twenty four weeks, at least six months, at least twenty six weeks, at least twenty eight weeks, at least seven months, at least thirty weeks, at least thirty two weeks, at least eight months, at least thirty four weeks, at lease thirty six weeks, at least nine months, at least thirty eight weeks, at least forty weeks, at least ten months, at least forty two weeks, at least forty four weeks, at least eleven months, at least forty six weeks, at least forty eight weeks, at least fifty weeks, at least fifty two weeks, at least twelve months, at least one year or more. In another embodiment, the method is suitable for short term, at least 8 days, to long term treatment. Contraceptive effect can be reversed by the subject by cessation of daily dosing.

In one embodiment, the daily dose comprises pellets or a dosage form comprising pellets as described herein with an amount of silodosin ranging from about 4 to 32 mg. In another embodiment, the daily dose comprises pellets or a dosage form comprising pellets as described herein with an amount of silodosin ranging from about 8 to 28 mg. In another embodiment, the daily dose comprises pellets or a dosage form comprising pellets as described herein with an amount of silodosin ranging from about 12 to 24 mg. In another embodiment, the particles are encompassed in a capsule, each capsule being filled with particles in a number sufficient to reach the daily dose.

Dosage forms of the modified release silodosin formulations described herein can contain one or more inactive ingredients. Inactive ingredients can include, but are not limited to water, buffers (including, by way of example and without limitation, phosphate buffers, citrate buffers, lactic acid, and others known to those of ordinary skill in the art), stabilizing agents (including, by way of example and without limitation, antioxidants (e.g., ascorbic acid, propionic acid, sodium bisulfite, sodium sulfite, and the like), chelating agents (e.g., fumaric acid, sodium edetate, and the like), and others known to those of ordinary skill in the art), surfactants (including, by way of example and without limitation, wetting agents (e.g., sorbitan monolaurate, etc.), antifoaming agents (e.g., sorbitan trioleate, etc.), detergents (e.g., sucrose stearate, etc.), solubilizing agents (e.g., polyethylene glycol 400 monostearate, etc.), and others known to those of ordinary skill in the art), processing aids (e.g., substances used to assist processing, including, by way of example and without limitation, lubricating agents, antioxidants, and others known to those of ordinary skill in the art), lubricating agents (including, by way of example and without limitation, stearic acid, calcium stearate, magnesium stearate, zinc stearate, talc, mineral and vegetable oils, benzoic acid, poly (ethylene glycol), glyceryl behenate. stearyl fumarate, and others known to those of ordinary skill in the art), emulsifiers (including, by way of example and without limitation, synthetic (e.g., sodium lauryl sulfate, potassium laurate, etc.), natural (e.g., gelatin, lecithin, etc.), and finely divided solid emulsifiers (e.g., bentonite, magnesium hydroxide, etc.), and others known to those of ordinary skill in the art), suspending agents (including, by way of example and without limitation, cellulose derivatives (e.g., carboxymethylcellulose, methylcellulose, ethyl cellulose, etc.), natural polymers (e.g., alginates, xanthan gum, guar gum, etc.), synthetic polymers (e.g., carbomers, polyvinyl pyrrolidone, etc.), clays (e.g., magnesium aluminum silicate, hectorite, etc.), and others known to those of ordinary skill in the art), preservatives (including, by way of example and without limitation, benzalkonium chloride, benzethonium chloride, benzyl alcohol, cetrimide, glycerin, propylene glycol, benzoic acid and sodium benzoate, potassium sorbate and sorbic acid, and others known to those of ordinary skill in the art), opaquing agents (including, by way of example and without limitation, titanium dioxide, and others known to those of ordinary skill in the art), glidants (including, by way of example and without limitation, silicon dioxide, colloidal or fumed silica, magnesium stearate, calcium stearate, stearic acid, cornstarch, talc and others known to those of ordinary skill in the art), diluents (including, by way of example and without limitation, corn syrup, lactose, sodium chloride, sucrose (sugar), and others known to those of ordinary skill in the art), colorants or coloring agents (including, by way of example and without limitation, FD&C Red No. 3. FD&C Red No. 20, FD&C Yellow No 6, FD&C Blue No. 2, D&C Green No. 5, FD&C Orange No 5, FD&C Red No. 8, caramel, ferric oxide, pigments, dyes, tints, titanium dioxide, natural coloring agents, such as grape skin extract, red beet powder, beta carotene, annatto, carmine, turmeric, paprika, black carrot juice, and others known to those of ordinary skill in the art), sweeteners or sweetening agents (including, by way of example and without limitation, sucrose, fructose, high fructose corn syrup, dextrose, saccharin sodium, maltodextrin, aspartame, potassium acesulfame, neohesperidin dihydrochalcone. sucralose, monoammonium glycyrrhizinate, and others known to those of ordinary skill in the art), perfuming agents (including, by way of example and without limitation, natural flavor oil, a synthetic flavor oil, and others known to those of ordinary skill in the art), glazing agents (including, by way of example and without limitation, vegetable oil, beeswax, carnauba wax, and others known to those of ordinary skill in the art), flavoring agents or flavorants (including, by way of example and without limitation, natural flavor oil, synthetic flavor oil, and other masking flavors known to those of ordinary skill in the art), and cooling agents (including, by way of example, N-substituted p-menthane-3-carboxamides, such as N-ethyl p-menthane-3-carboxamide (“WS-3”) (Millennium Specialty Chemicals, Jacksonville, Fla.) Additional examples of other inactive ingredients are well known in the art. See, e.g., REMINGTON: THE SCIENCE AND PRACTICE OF PHARMACY (21st ed).

In one embodiment, the composition does not impair erectile function of the male subject. In another embodiment, the composition does not impair the quality of orgasm of the male subject. In another embodiment, the composition does not cause discomfort upon ejaculation for the male subject. Orgasm quality can be measured by, e.g., the Orgasm Rating Scale, ORS, for the quality of orgasm (see, e.g., Mah, K., & Binik, Y. M. (2020). Orgasm Rating Scale. In R. R. Milhausen, J. K. Sakaluk, T. D. Fisher, D. M. Davis & W. L. Yarber (Eds.), Handbook of Sexuality-Related Measures (4th ed., pp. 503-507). Routledge).

Doses can be packaged to include, for example, at least 7, 14, 28, 56, 84, 168 to 365 unitary doses; or 10, 20, 30, 60, 90, or 180 to 360 unitary doses of a composition as described herein, each unitary dose being a daily dose.

In one embodiment, described herein is a method for reversible, continuous, non-hormonal contraception in a male subject, the method comprising administering once daily doses of a composition comprising: an modified release formulation of silodosin as described herein in an amount effective, when administered on a once daily dosing regimen, to induce a reversible condition of aspermia, azoospermia or severe oligozoospermia sufficient for contraceptive effect in the male subject, wherein after an initial period of at least two consecutive days, the continuous state of aspermia, azoospermia or severe oligozoospermia sufficient for contraceptive effect is not impaired by a delay of the subsequent once daily intake.

In another embodiment, described herein is a method for birth control, the method comprising administering to a male subject once daily doses of a composition comprising: an modified release formulation of silodosin as described herein in an amount effective, when administered on a once daily dosing regimen, to induce a reversible condition of aspermia, azoospermia or severe oligozoospermia sufficient for contraceptive effect in the male subject, wherein after an initial period of at least two consecutive days, the continuous state of aspermia, azoospermia or severe oligozoospermia sufficient for contraceptive effect is not impaired by a delay of the subsequent once daily intake.

According to some embodiments, the method or the use is non-therapeutic.

According to some embodiments, the method is non-hormonal or the use is for non-hormonal contraception. “Non-hormonal” means that no hormone, in particular no male hormone, is administered to the subject in the course of the method or use.

According to some embodiments, the method or the use comprises a step of administering to a male subject an effective amount of a composition as described herein.

“Effective amount” refers to the amount of an active ingredient (e.g., silodosin) that is sufficient to achieve the desired therapeutic, prophylactic or preventative effect (e.g., contraception), in the subject to which/whom it is administered, without causing significant negative or adverse side effects to said subject.

According to some embodiments, the composition is administered, or is to be administered, to said male subject at about the same time each day.

“About the same time” means plus or less (plus or minus or more or less) two hours (±2 h).

### Manufacturing Process

The pellet or the plurality of pellets as described herein can be manufactured by means of coating methods know in the art such as, for example, spray coating.

Another object of the technology described herein is a process for manufacturing a plurality of pellets or a composition as described herein.

According to some embodiments, the process comprises the following steps:
(1-a) preparing a drug solution or drug suspension comprising
   a drug solution comprising silodosin, at least one binder, at least one solvent, and, optionally, at least one antioxidant; and
   optionally, at least one anti-tacking and/or charge agent, then
(1-b) applying the drug solution or drug suspension onto a plurality of inert cores, thereby obtaining a plurality of pellets A;
(2-a) optionally, preparing a sealing coating suspension comprising at least one sealing coating agent; then
(2-b) applying the sealing coating suspension onto the plurality of pellets A, thereby obtaining a plurality of pellets B; and
applying the extended-release coating and, optionally, the enteric coating, onto the plurality of pellets A or pellets B, typically as described hereinafter.

According to some preferred embodiments, the process further comprises the following steps:

(3-a) preparing an extended-release coating suspension comprising at least one extended-release coating agent, at least one plasticizer, and at least one anti-tacking agent; then
(3-b) applying the extended-release coating suspension onto the plurality of pellets A or onto the plurality of optional pellets B, thereby obtaining the plurality of pellets (“pellets C”).

According to some preferred embodiments, the process further comprises the following steps:
(3′-a) preparing an extended-release coating suspension comprising at least one extended-release coating agent, at least one plasticizer, and at least one anti-tacking agent; then
(3′-b) applying the extended-release coating suspension onto the plurality of pellets A or onto the plurality of optional pellets B, thereby obtaining a plurality of pellets C;
(4′-a) preparing an enteric coating suspension comprising at least one enteric coating agent and at least one plasticizer; then
(4′-b) applying the enteric coating suspension onto the plurality of pellets C, thereby obtaining the plurality of pellets (“pellets D”).

According to other embodiments, the process further comprises the following steps:
(3″-a) preparing an enteric coating suspension comprising at least one enteric coating agent and at least one plasticizer; then
(3″-b) applying the enteric coating suspension onto the plurality of pellets A or onto the plurality of optional pellets B, thereby obtaining a plurality of pellets E;
(4″-a) preparing an extended-release coating suspension comprising at least one extended-release coating agent, at least one plasticizer, and at least one anti-tacking agent; then
(4″-b) applying the extended-release coating suspension onto the plurality of pellets E, thereby obtaining the plurality of pellets (“pellets F”).

According to some embodiments, at least one among the drug layer and the coatings is applied by spraying. In some embodiments, the drug layer and each coating are applied by spraying. The application can be carried out by spray coating methods well-known in the art.

According to some embodiments, the solvent in the drug solution or suspension at step (1-a) is ethanol.

According to some embodiments, the process further comprises the following step: (5) filling of the obtained plurality of pellets (“pellets C”, “pellets D” or “pellets F” depending on the previous steps) into at least one container, thereby obtaining a dosage form as described herein.

According to some embodiments, the manufactured composition is a dosage form. According to some embodiments, the container filled in step (5) is a capsule.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in pharmaceutical or pharmacological use can be found in The Merck Manual of Diagnosis and Therapy, 20th Edition, published by Merck Sharp & Dohme Corp., 2018 (ISBN 0911910190, 978-0911910421); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, Ada M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims. Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The technology described herein is further illustrated by the following non-limiting examples.

### Example 1: Silodosin Formulations

### Example 1-1: Formulation (I-a)

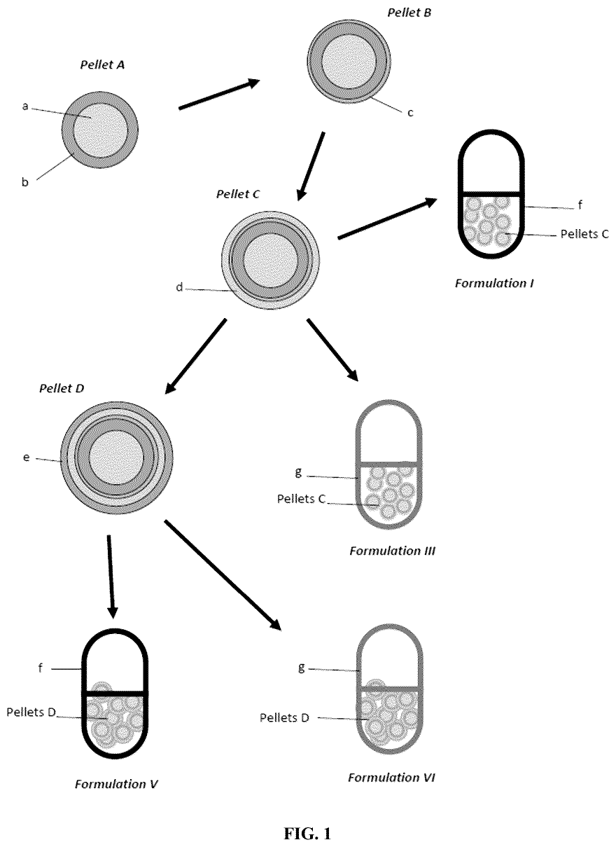

Modified-release silodosin pellets prepared as follows (cf. FIG. 1 for schematic representation of the manufacturing process): (R)-Silodosin was dissolved in an ethanolic solution of Hydroxypropylcellulose (HPC) and butylated hydroxytoluene (BHT), thereby obtaining a silodosin solution. Calcium carbonate (CaCO₃) was then added to the solution, thereby obtaining a drug layering suspension. The composition of the layering (R)-Silodosin suspension is detailed in Table 2.

TABLE 2
Composition of the layering (R)-Silodosin suspension

| Ingredient | % w/w |
|---|---|
| (R)-Silodosin | 9.19 |
| HPC | 0.92 |
| CaCO₃ | 4.60 |
| BHT | 0.29 |
| Ethanol | 85.0 |

Then, the layering (R)-Silodosin suspension was sprayed (bottom spray) under continuous stirring onto inert cores (cellulose microspheres). Initial parameters were set as follows: fluidization inlet air temperature: 56.0° C., air flow rate: 70 m³/h. The composition of the obtained Pellets A-I-a is detailed in Table 3.

TABLE 3
Composition of Pellets A-I-a

| Ingredient | % w/w |
|---|---|
| (R)-Silodosin | 13.0 |
| HPC | 1.3 |
| CaCO₃ | 6.5 |
| BHT | 0.4 |
| Cellulose microspheres | 78.8 |

An aqueous solution of Hydroxypropylmethylcellulose (HPMC) [Opadry Clear 03A6900067®] was then sprayed onto the Pellets A-I-a previously prepared. Initial parameters were set as follows: fluidization inlet air temperature: 54.0° ° C., air flow rate: 70 m³/h. The composition of the obtained Pellets B-I-a is presented in Table 4.

TABLE 4
Composition of Pellets B-I-a

| Ingredient | % w/w |
|---|---|
| Pellets A-I-a | 95 |
| HPMC | 5 |

The coated Pellets B-I-a previously prepared were further coated by spraying with an aqueous suspension of polyvinyl acetate [Kollicoat® SR 30 D (KSR)] (14.6% w/w), Triethylcitrate (TEC) (0.73% w/w) and talc (5.1% w/w) maintained under continuous stirring. Initial parameters were set as follows: fluidization inlet air temperature: 43.0° C., air flow rate: 75 m³/h. Then, the coated pellets were cured for 120 min at 45° C. The composition of the obtained Pellets C-I-a is presented in Table 5 and Table 6.

TABLE 5
Composition of Pellets C-I-a

| Ingredient | % w/w |
|---|---|
| Pellets B-I-a | 85.00 |
| Kollicoat ® SR 30 D | 10.71 |
| TEC | 0.54 |
| Talc | 3.75 |

TABLE 6
Composition of lubricated Pellets C-I-a

| | Unit formula | Quantity per |
|---|---|---|
| Ingredient | (% w/w) | capsule (mg) |
| (R)-Silodosin | 10.45 | 12.00 |
| Cellulose microspheres | 63.31 | 72.73 |
| HPC | 1.04 | 1.20 |
| BHT | 0.33 | 0.38 |
| CaCO₃ | 5.22 | 6.00 |
| HPMC | 4.23 | 4.86 |
| TEC | 0.53 | 0.61 |
| Talc [in pellets] | 3.73 | 4.29 |
| Kollicoat ® SR 30 D | 10.66 | 12.24 |
| Talc [as lubricant] | 0.50 | 0.57 |

Hypromellose hard capsules were filled with the adequate quantity of Pellets C-I-a previously prepared and lubricated with 0.50% w/w of talc, to a final (R)-Silodosin content of 12 mg per capsule, thereby obtaining Formulation (I-a).

### Example 1-2: Formulation (V-a)

Modified-release silodosin pellets prepared as follows (cf. FIG. 1 for schematic representation of the manufacturing process): (R)-Silodosin was dissolved in an ethanolic solution of Hydroxypropylcellulose (HPC) and butylated hydroxytoluene (BHT), thereby obtaining a silodosin solution. Calcium carbonate (CaCO₃) was then added to the solution, thereby obtaining a silodosin suspension. The composition of the layering (R)-Silodosin suspension is detailed in Table 7.

TABLE 7
Composition of the layering (R)-Silodosin suspension

| Ingredient | % w/w |
|---|---|
| (R)-Silodosin | 9.19 |
| HPC | 0.92 |
| CaCO₃ | 4.60 |
| BHT | 0.29 |
| Ethanol | 85.0 |

Then, the layering (R)-Silodosin suspension was sprayed (bottom spray) under continuous stirring onto inert cores (cellulose microspheres). Initial parameters were set as follows: fluidization inlet air temperature: 56.0° C., air flow rate: 70 m³/h. The composition of the obtained Pellets A-V-a is detailed in Table 8.

TABLE 8
Composition of Pellets A-V-a

| Ingredient | % w/w |
|---|---|
| (R)-Silodosin | 13.0 |
| HPC | 1.3 |
| CaCO₃ | 6.5 |
| BHT | 0.4 |
| Cellulose microspheres | 78.8 |

An aqueous solution of Hydroxypropylmethylcellulose (HPMC) [Opadry Clear 03A6900067®] was then sprayed onto the Pellets A-V-a previously prepared. Initial parameters were set as follows: fluidization inlet air temperature: 54.0° C., air flow rate: 70 m³/h. The composition of the obtained Pellets B-V-a is presented in Table 9.

TABLE 9
Composition of Pellets B-V-a

| Ingredient | % w/w |
|---|---|
| Pellets A-V-a | 95 |
| HPMC | 5 |

The coated Pellets B-V-a previously prepared were further coated by spraying with an aqueous suspension of polyvinyl acetate [Kollicoat® SR 30 D (KSR)] (14.6% w/w), Triethylcitrate (TEC) (0.73% w/w) and talc (5.1% w/w) maintained under continuous stirring. Initial parameters were set as follows: fluidization inlet air temperature: 43.0° C., air flow rate: 75 m³/h. Then, the coated pellets were cured for 120 min at 45° C. The composition of the obtained Pellets C-V-a is presented in Table 10.

TABLE 10
Composition of Pellets C-V-a

| Ingredient | % w/w |
|---|---|
| Pellets B-V-a | 92.50 |
| Kollicoat ® SR 30 D | 5.36 |
| TEC | 0.26 |
| Talc | 1.88 |

A final coating was applied to Pellets C-V-a previously prepared by spraying thereon an aqueous suspension of Methacrylic acid ethyl acrylate copolymer [Kollicoat MAE 30 DP® (KMAE)] (60.6% w/w) and Triethylcitrate (TEC) (1.82% w/w) maintained under continuous stirring. Initial parameters were set as follows: fluidization inlet air temperature: 41° C., air flow rate: 70 m³/h. The composition of the obtained Pellets D-V-a is presented in Table 11 and Table 12.

TABLE 11
Composition of Pellets D-V-a

| Ingredient | % w/w |
|---|---|
| Pellets C-V-a | 75.0 |
| TEC | 2.27 |
| Kollicoat MAE 30 DP ® | 22.7 |

TABLE 12
Composition of lubricated Pellets D-V-a

| | Unit formula | Quantity per |
|---|---|---|
| Ingredient | (% w/w) | capsule (mg) |
| (R)-Silodosin | 8.52 | 12.00 |
| Cellulose microspheres | 51.67 | 72.73 |
| HPC | 0.85 | 1.20 |
| BHT | 0.27 | 0.38 |
| CaCO₃ | 4.26 | 6.00 |
| HPMC | 3.45 | 4.86 |
| TEC | 2.46 | 3.46 |
| Talc [in pellets] | 1.40 | 1.97 |
| Kollicoat ® SR 30 D | 4.00 | 5.63 |
| Kollicoat MAE 30 DP ® | 22.61 | 31.83 |
| Talc [as lubricant] | 0.50 | 0.70 |

Hypromellose hard capsules were filled with the adequate quantity of Pellets D-V-a previously prepared lubricated with 0.50% w/w of talc, to a final (R)-silodosin content of 12 mg per capsule, thereby obtaining Formulation (V-a).

### Example 2: In Vitro Studies of Silodosin Formulations

### Example 2-1: Formulation (I-a)

The dissolution tests were each conducted according to US Pharmacopeia method, at 50 rpm in 900 mL of 0.1 N HCl (acidic medium) solution or pH 6.8 phosphate buffer (neutral medium) in a USP type 2 apparatus at 37° C.±0.5° C., with direct UV detection.

The dissolution profile of Formulation (I-a) at pH 6.8 is presented on FIG. 2, which shows that 85% of the silodosin amount is released in about 34 hours from Formulation (I-a) and in about 10 min from the comparative IR composition (RAPAFLO®), and that the release rate of Formulation (I-a) is much more slower than the release rate of the comparative IR composition (RAPAFLO®) as evidenced by the slopes of their respective dissolution profiles. Therefore, Formulation (I-a) is a modified-release formulation, in sharp contrast with the comparative IR composition (RAPAFLO®). A “lag time” of about one hour appears on FIG. 2, which shows that Formulation (I-a) is a delayed-release formulation.

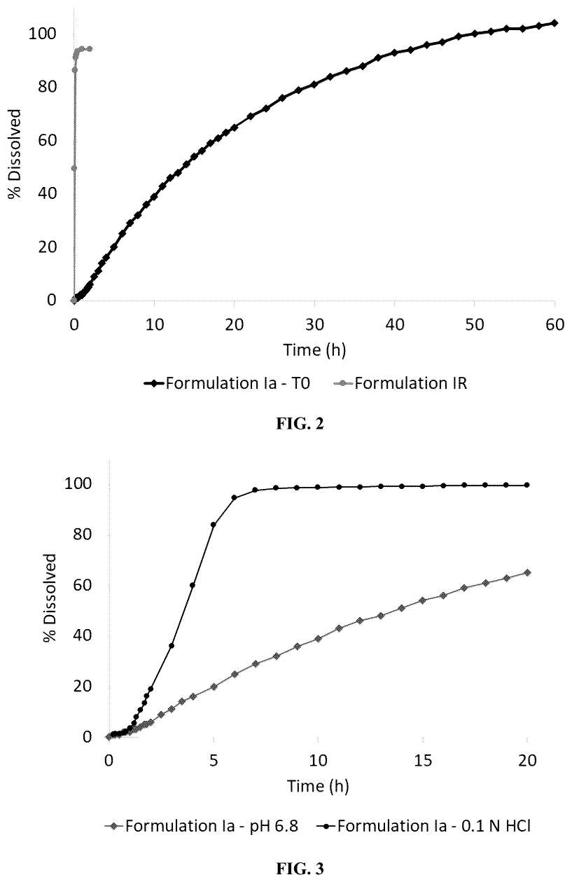

The dissolution profile of Formulation I-A in 0.1 N HCl and at pH 6.8 is presented on FIG. 3 and shows that 50% of silodosin is released in about 14 hours in pH 6.8 and in about 3 hours in 0.1 N HCl and the release rate is much more slower in pH 6.8 than in 0.1 N HCl, as evidenced by the slopes of the dissolution profiles. The dissolution profile of Formulation I-a is therefore “pH-dependent” with a faster release rate in the acidic medium.

Table 13 below shows the chemical stability of silodosin in Formulation (I-a) over time (To, after 3 months, after 9 months and after 18 months) in two different storage conditions: at 40° C. and 75% of relative humidity (RH) or 25° C. and 60% of relative humidity (RH). Dehydrosilodosin is the main degradation product of silodosin.

TABLE 13
Silodosin, dehydrosilodosin and total silodosin degradation
products content in Formulation (I-a) over time

| | | Dehydrosilodosin | Total degradation |
|---|---|---|---|
| | Silodosin | content | products content |
| | content | (% w/w of the | (% w/w of the |
| | (mg/capsule) | dose) | dose) |
| T0 | 12.2 | ≤0.1 | ≤0.1 |
| T3 months at | 12.1 | 0.2 | 0.2 |
| 40° C./7% RH | | | |
| T9 months at | 12.2 | 0.2 | 0.2 |
| 25° C./60% RH | | | |
| T18 months at | 12.1 | 0.2 | 0.2 |
| 25° C./60% RH | | | |

Table 13 confirms the chemical stability of silodosin in Formulation (I-a) over time in both storage conditions, since no significant decrease of the silodosin content and no significant increase in degradation products content is observed.

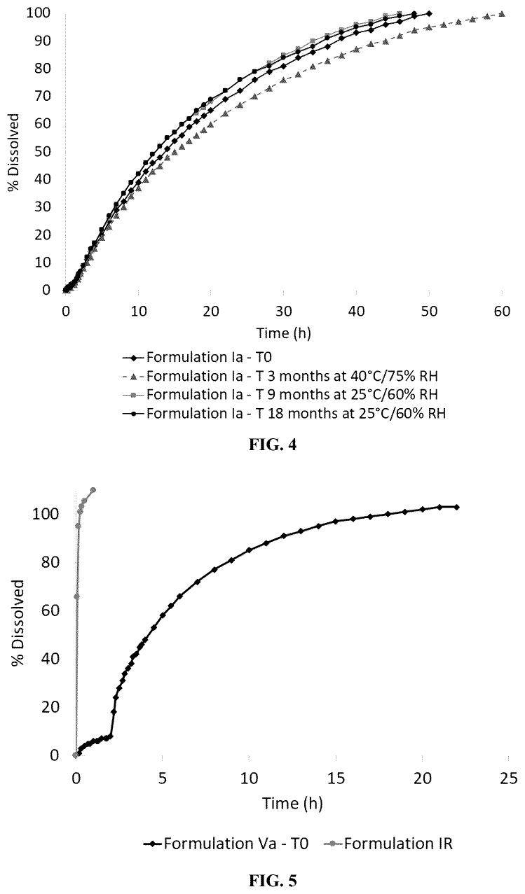

FIG. 4 shows that the dissolution profiles of Formulation (I-a) at TO, after 3 months of storage at 40° C. and 75% RH, after 9 months of storage at 25° C. and 60% RH and after 18 months of storage at 25° C. and 60% RH are almost superimposed, demonstrating that the dissolution profile of Formulation (I-a) is not affected over time by storage (in both storage conditions).

### Example 2-2: Formulation (V-a)

The dissolution tests were each conducted according to US Pharmacopeia method, at 50 rpm in 900 mL of simulated gastric juice during 2 hours and then in pH 6.8 phosphate buffer (which is referred to as “progressive pH medium”, which is recommended by USP for enteric formulation) in a USP type 2 apparatus at 37° C.±0.5° C., with direct UV detection.

The dissolution profile of Formulation (V-a) in the progressive pH medium is presented on FIG. 5, which shows that 85% of the silodosin amount is released in about hours from Formulation (V-a) and in less than 10 min from the comparative IR composition (RAPAFLO®), and that the release rate of Formulation (V-a) is much more slower than the release rate of the comparative IR composition (RAPAFLO®) as evidenced by the slopes of their respective dissolution profiles. Therefore, Formulation (V-a) is a modified-release formulation, in sharp contrast with the comparative IR composition (RAPAFLO®). A delay of about two hours appears on FIG. 5, which shows that Formulation (V-a) is a delayed-release formulation.

Table 14 below shows the chemical stability of silodosin in Formulation (V-a) over time (To, after 3 months and after 9 months) in two different storage conditions: at 40° C. and 75% of relative humidity (RH) or 25° C. and 60% of relative humidity (RH). Dehydrosilodosin is the main degradation product of silodosin.

TABLE 14
Silodosin, dehydrosilodosin and total silodosin degradation
products content in Formulation (V-a) over time

| | | Dehydrosilodosin | Total degradation |
|---|---|---|---|
| | Silodosin | content | products content |
| | content | (% w/w of the | (% w/w of the |
| | (mg/capsule) | dose) | dose) |
| T0 | 11.9 | ≤0.1 | ≤0.1 |
| T3 months at | 11.6 | 0.2 | 0.2 |
| 40° C./7% RH | | | |
| T9 months at | 11.9 | 0.2 | 0.2 |
| 25° C./60% RH | | | |

Using a different batch of Formulation (V-a), prepared exactly as described in Example 1-2 hereinabove, chemical stability of silodosin in Formulation (V-a) was also confirmed to last up to 12 months at 25° C./60% RH (data not shown).

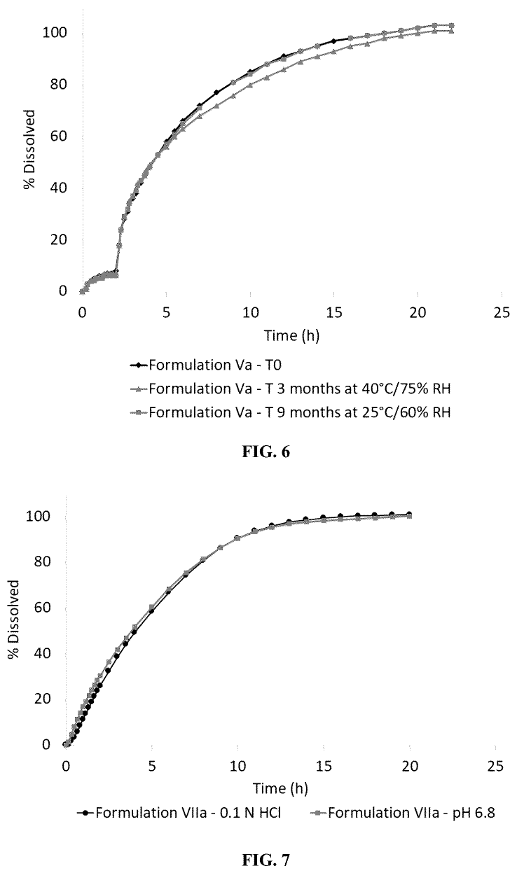

FIG. 6 shows that the dissolution profile of Formulation (V-a) is not affected over time by storage (in both storage conditions), since the dissolution profiles at T₀, 3 months and 9 months are very similar.

### Example 3: In Vitro Study of Comparative Silodosin Formulations

### Example 3-1: Ethyl Cellulose

### Example 3-1-1: Aquacoat® ECD 30 (Test 1)

The manufacturing process of Comparative Formulation (VII-a) was essentially as described in Example 1-1 for Formulation (I-a), except that BHT was replaced by another antioxidant alpha-tocopherol, thereby obtaining the pellets A-VII-a with the composition shown in Table 15.

In addition, the coating apply to pellets B-VII-a was a suspension containing 12% w/w of ethylcellulose and 3% of dibutylsebaccate (DBS), then the granules were cured for 4 h at 60° C. with simultaneous water spraying, thereby obtaining the Comparative Pellets C-VII-a with the composition shown in Table 16.

TABLE 15
Composition of pellets A-VII-a

| Ingredient | % w/w |
|---|---|
| Silodosin | 13.000 |
| HPC | 1.300 |
| CaCO₃ | 6.500 |
| Alpha-tocopherol | 0.142 |
| Cellulose microsphere | 79.058 |

TABLE 16
Composition of Comparative Pellets C-VII-a

| Ingredient | % w/w |
|---|---|
| Pellets B-VII-a | 85% |
| Ethylcellulose | 12% |
| DBS | 3% |

Hypromellose hard capsules were filled with the adequate quantity of Comparative Pellets C-VII-a previously prepared to a final (R)-silodosin content of 12 mg per capsule, thereby obtaining Comparative Formulation (VII-a).

The dissolution test was conducted according to US Pharmacopeia method, at 50 rpm in 900 mL of 0.1 N HCl solution or a pH 6.8 phosphate buffer in a USP type 2 apparatus at 37° C.±0.5° C., with direct UV detection.

The dissolution profile of Comparative Formulation (VII-a) in HCl 0.1 and at pH 6.8 is presented on FIG. 7, which shows that Comparative Formulation (VII-a) provided a somewhat extended-release profile with 85% of silodosin released in about 9 hours. FIG. 7 also shows that this dissolution profile of Comparative Formulation (VII-a) is pH-independent since the two curves are almost superimposed.

### Example 3-1-2: Aquacoat® ECD 30 (Test 2)

The manufacturing process of Comparative Pellets (VII-b) was essentially as described in Example 3-1-1 for Comparative Formulation (VII-a), except that the pellets were cured for 2 h at 60° C. without water spraying, thereby obtaining the Comparative Pellets C-VII-b with the composition shown in Table 17.

TABLE 17
Composition of Comparative Pellets C-VII-b

| Ingredient | % w/w |
|---|---|
| Pellets B-VII-a | 85% |
| Ethylcellulose | 12% |
| DBS | 3% |

The dissolution test was conducted according to US Pharmacopeia method, at 50 rpm in 900 mL of 0.1 N HCl solution in a USP type 2 apparatus at 37° C.±0.5° C., with direct UV detection.

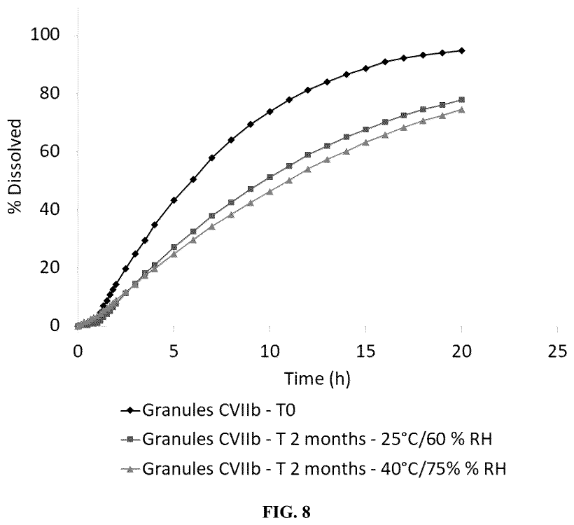

The dissolution profiles of Comparative Pellets (VII-b) in 0.1 N HCl solution are presented on FIG. 8, which shows that the dissolution profile of Comparative Pellets (VII-b) after 2 months of storage at 25° C. and 60% RH is much slower than at TO and even more slower after 2 months of storage at 40° C. and 65% RH. The dissolution profile of Comparative Pellets (VII-b) is therefore not stable over time.

Therefore, Comparative Pellets C-VII-b are not suitable for use in continuous contraception methods.

### Example 3-1-3: Surelease®

Another commercial aqueous suspension of ethylcellulose (Surelease®, which is an ethylcellulose in suspension with a plurality additives) was tested as ER coating. Pellets were manufactured with a weight gain of about 25% of ethylcellulose.

The Applicant found that degradation of silodosin in these pellets was very high, far beyond acceptable working range. Without being bound by theory, the Applicant suspects a chemical incompatibility between silodosin and at least one component of Surelease®.

### Example 3-2: Polymethacrylates

The manufacturing process of Comparative Pellets C-VIII-a and C-IX-a was essentially as described in Example 1-1 for Pellets C-I-a, except that the final ER coating is either
(Pellets C-VIII-a) a suspension containing a neural copolymer of ethyl acrylate and methyl methacrylate (commercially available under tradename Eudragit® NM 30 D, Evonik Corporation, Germany), hydroxypropylmethylcellulose (HPMC), polysorbate 80 and talc; or
(Pellets C-IX-a) copolymer a copolymer of ethyl acrylate, methyl methacrylate and a low content of methacrylic acid ester with quaternary ammonium groups (commercially available under tradename Eudragit® RS 100, Evonik Corporation, Germany), triethylcitrate (TEC) and talc.
   The composition of the obtained Comparative Pellets C-VIII-a is shown in Table 18.

TABLE 18
Composition of Comparative Pellets C-VIII-a

| Ingredient | % w/w |
|---|---|
| Pellets B-VIII-a | 80.0% |
| Eudragit ® NM 30D | 9.1% |
| HPMC | 0.9% |
| Polysorbate 80 | 0.9% |
| Talc | 9.1% |

The compositions of the obtained Comparative Pellets C-IX-a are shown on Table 19.

TABLE 19
Composition of Comparative Pellets C-IX-a

| Ingredient | % w/w |
|---|---|
| Pellets B-IX-a | 85.0% |
| Eudragit ® RS 100 | 9.4% |
| TEC | 0.9% |
| Talc | 4.7% |

The dissolution test was conducted according to US Pharmacopeia method, at 50 rpm in 900 mL of 0.1 N HCl solution or a pH 6.8 phosphate buffer in a USP type 2 apparatus at 37° C.±0.5° C., with direct UV detection.

With Comparative Pellets C-VIII-a, the release profile is almost immediate-release. In about one hour, 100% of silodosin is released.

With Comparative Pellets C-IX-a, the release profile is biphasic (“S-shaped”) with a first phase of very slow release from 0 to about 6 h, followed by a rapid phase from about 6 h to about 11 h. At this time, 100% of silodosin is released.

Therefore, Comparative Pellets C-VIII-a are not suitable for use in continuous contraception methods. Moreover, Comparative Pellets C-IX-a are not advantageous for use as ER composition in continuous contraception methods.

### Example 4: In Vivo Study of Silodosin Formulations

### Materials and Methods

A study was conducted to evaluate the pharmacokinetic (PK) profiles of the formulations described herein in the treatment of male subjects. A total of 24 subjects aged 20 to 48 years were enrolled in a double-blind study. Each subject receives single doses of formulation (I-a) and formulation (V-a) in a cross-over design. Plasma samples were collected from 0 to 48 hours post-dose for silodosin determination.

The potential for a burst on the PK profiles when administering the formulations was assessed.

### Results

No burst was observed on the PK profiles, which evidences that formulation (I-a) and formulation (V-a) are not subject to any significant burst release effect when administered to male subjects.

1. A pellet comprising:
(a) an inert core;
(b) at least one drug layer applied to the inert core, the at least one drug layer comprising:
silodosin, and
at least one binder; and
(c) at least one extended-release coating surrounding the at least one drug layer, wherein said at least one extended-release coating comprises at least one vinyl polymer.

2. The pellet of claim 1, further comprising at least one sealing coating surrounding the at least one drug layer, and wherein the at least one extended-release coating surrounds the sealing coating.

3. The pellet of claim 1, wherein the at least one binder comprises a cellulose polymer.

4. The pellet of claim 3, wherein the cellulose polymer comprised by the at least one binder is selected from the group consisting of:
hydroxypropyl cellulose (HPC), hydroxypropyl methylcellulose (HPMC), carboxymethylcellulose, methylcellulose, ethyl cellulose, povidone, polyvinylpyrrolidone, and mixtures thereof.

5. The pellet of claim 2, wherein the at least one sealing coating comprises at least one cellulose polymer.

6. The pellet of claim 5, wherein the cellulose polymer comprised by the at least one sealing coating is selected from the group consisting of:
hydroxypropyl methylcellulose (HPMC), carboxymethylcellulose, methylcellulose, ethyl cellulose, and mixtures thereof.

7. The pellet of claim 1, wherein the at least one extended-release coating comprises at least one of:
at least one polyvinyl ester polymer, and
povidone (PVP).

8. The pellet of claim 7, wherein the at least one polyvinyl ester polymer is a polyvinyl acetate polymer.

9. The pellet of claim 8, wherein the polyvinyl acetate polymer is polyvinyl acetate (PVA).

10. (canceled)

11. The pellet of claim 9, wherein the at least one extended-release coating comprises about 90% w/w of polyvinyl acetate (PVA) and about 9% w/w of povidone (PVP), in weight by weight of the at least one extended-release coating.

12. The pellet of claim 2, wherein the pellet further comprises one or more of:
an antioxidant;
an anti-tacking and/or charge agent; and/or
one or more plasticizer.

13. The pellet of claim 12, wherein the antioxidant is selected from the group consisting of:
phenols, vitamin E and derivatives thereof, vitamin C and derivatives thereof, propyl gallate, butylated hydroxytoluene (BHT), butylated hydroxyanisole (BHA), alpha-tocopherol, ascorbyl palmitate, and mixtures thereof.

14. The pellet of claim 12, wherein the anti-tacking and/or charge agent is selected from the group consisting of:
inorganic carbonates, calcium carbonate (CaCO₃), magnesium silicates, talc, and mixtures thereof.

15. The pellet of claim 12, wherein the plasticizer is a citric acid ester.

16. The pellet of claim 15, wherein the plasticizer is triethyl citrate (TEC).

17. The pellet of claim 1, wherein the pellet further comprises at least one enteric coating, wherein the enteric coating is either:
surrounding the at least one extended-release coating, or
surrounded by the at least one extended-release coating and surrounding the at least one sealing coating or the at least one drug layer.

18. The pellet of claim 17, wherein the enteric coating comprises at least one acrylate copolymer.

19. The pellet of claim 18, wherein the acrylate copolymer is a methacrylic acid ethylacrylate (MAE) copolymer.

20. The pellet of claim 1, wherein the pellet comprises:
a pellet A consisting essentially of:
from about 24 to 95% w/w of the inert core;
from about 5 to 76% w/w of the at least one drug layer, the at least one drug layer comprising:
from about 5 to 25% w/w of the silodosin,
from about 0.1 to 7.5% w/w of the at least one binder,
from about 0 to 20% w/w of at least one antioxidant, and
from about 0 to 25% w/w of at least one anti-tacking and/or charge agent, in weight relative to the total weight of said pellet A.

21. The pellet of claim 20, wherein the pellet A is comprised in a pellet B consisting essentially of:
from about 90 to 100% w/w of the pellet A, and
from about 0 to 10% w/w of at least one sealing coating,
in weight relative to the total weight of the pellet B.

22. The pellet of claim 21, wherein the pellet B is comprised in a pellet C consisting essentially of:
from about 50 to 98% w/w of the pellet B, and
an extended-release coating comprising:
from about 0.5 to 47% w/w of at least one extended-release agent,
from about 0.02 to 7% w/w of at least one plasticizer, and
from about 0.3 to 23% w/w of at least one anti-tacking agent, in weight relative to the total weight of the pellet C.

23. The pellet of claim 22, wherein the pellet C is comprised in a pellet D consisting essentially of:
from about 50 to 95% w/w of the pellet C, and
an enteric coating comprising:
from about 4 to 49.5% w/w of at least one enteric agent, and
from about 0.04 to 10% w/w of at least one plasticizer,
in weight relative to the total weight of the pellet D.

24. The pellet of claim 1, wherein:
(a) the at least one drug layer is applied onto the inert core at a weight gain ranging from about 5 to 318%,
(b) at least one sealing coating agent is applied onto the at least one drug layer at a weight gain ranging from 0 to about 11%,
(c) the at least one extended-release coating agent is applied onto the at least one drug layer or at least one sealing coating at a weight gain ranging from about 2 to 100%, and/or
(d) at least one enteric coating agent is applied onto the at least one extended-release coating at a weight gain ranging from 0 to about 100%.

25. The pellet of claim 1, wherein said pellet comprises:
(a) one inert core comprising cellulose microspheres;
(b) at least one drug layer applied to the inert core, wherein the at least one drug layer comprises:
silodosin,
hydroxypropyl cellulose (HPC),
calcium carbonate (CaCO₃), and
butylated hydroxytoluene (BHT);
(c) at least one sealing coating surrounding the at least one drug layer, wherein the at least one sealing coating comprises hydroxypropyl methylcellulose (HPMC); and
(d) at least one extended-release coating surrounding the at least one drug layer or said optional the at least one sealing coating, wherein the at least one extended-release coating comprises:
polyvinyl acetate (PVA),
polyvinylpyrrolidone (PVP),
triethyl citrate (TEC), and
talc.

26. The pellet of claim 25, wherein the pellet further comprises:
(e) at least one enteric coating, wherein the at least one enteric coating is either surrounding the at least one extended-release coating, or surrounded by the at least one extended-release coating and surrounding the at least one sealing coating or the at least one drug layer, wherein said enteric coating comprises:
a methacrylic acid ethylacrylate copolymer 1:1 (MAE 1:1), and
triethyl citrate (TEC).

27. A pharmaceutical composition comprising a plurality of pellets according to claim 1.

28. The pharmaceutical composition of claim 27, wherein the plurality of pellets is comprised in at least one capsule; wherein the at least one capsule is selected from the group consisting of a hard-shell capsule, a functional capsule, and an enteric capsule.

29. (canceled)

30. (canceled)

31. A contraception method for a male subject comprising administering a pharmaceutical composition of claim 27 to the male subject at about the same time each day.

32. A method of inducing a state of reversible aspermia, azoospermia, or severe oligozoospermia in a male subject sufficient for contraceptive effect, the method comprising administering a pharmaceutical composition of claim 27 to the male subject at about the same time each day.

33. A process for manufacturing a plurality of pellets according to claim 1, wherein said process comprises the following steps:
(1-a) preparing a drug solution or drug suspension comprising
a drug solution comprising silodosin, at least one binder and at least one solvent;
(1-b) applying said drug solution or drug suspension onto a plurality of inert cores, thereby obtaining a plurality of pellets A;
(2-a) applying a sealing coating suspension onto said plurality of pellets A, thereby obtaining a plurality of pellets B;
(3-a) preparing an extended-release coating suspension comprising at least one extended-release coating agent, at least one plasticizer, and at least one anti-tacking agent; then
(3-b) applying said extended-release coating suspension onto said plurality of pellets A or onto said plurality of pellets B, thereby obtaining the plurality of pellets according to claim 1.

34. (canceled)

35. (canceled)

36. (canceled)

37. A process for manufacturing a plurality of pellets according to claim 1, wherein said process comprises the following steps:
(1-a) preparing a drug solution or drug suspension comprising
a drug solution comprising silodosin, at least one binder and at least one solvent;
(1-b) applying said drug solution or drug suspension onto a plurality of inert cores, thereby obtaining a plurality of pellets A;
(2-a) applying a sealing coating suspension onto said plurality of pellets A, thereby obtaining a plurality of pellets B;
(3-a) preparing an extended-release coating suspension comprising at least one extended-release coating agent, at least one plasticizer, and at least one anti-tacking agent; then (3′-b) applying said extended-release coating suspension onto said plurality of pellets A or onto said plurality of pellets B, thereby obtaining a plurality of pellets C;
(4′-a) preparing an enteric coating suspension comprising at least one enteric coating agent and at least one plasticizer; then
(4′-b) applying said enteric coating suspension onto said plurality of pellets C, thereby obtaining the plurality of pellets according to claim 1.

38. (canceled)

39. (canceled)

40. (canceled)

41. A process for manufacturing a plurality of pellets according to claim 1, wherein said process comprises the following steps:
(1-a) preparing a drug solution or drug suspension comprising
a drug solution comprising silodosin, at least one binder and at least one solvent;
(1-b) applying said drug solution or drug suspension onto a plurality of inert cores, thereby obtaining a plurality of pellets A;
(2-a) applying a sealing coating suspension onto said plurality of pellets A, thereby obtaining a plurality of pellets B;
(3″-a) preparing an enteric coating suspension comprising at least one enteric coating agent and at least one plasticizer; then (3″-b) applying said enteric coating suspension onto said plurality of pellets A or onto said plurality of pellets B, thereby obtaining a plurality of pellets E;
(4″-a) preparing an extended-release coating suspension comprising at least one extended-release coating agent, at least one plasticizer, and at least one anti-tacking agent; then
(4″-b) applying said extended-release coating suspension onto said plurality of pellets E, thereby obtaining the plurality of pellets according to claim 1.

42.-45. (canceled)
